(19) United States
(12) Patent Application Publication
(10) Pub. No.: US 2022/0389524 A1
(43) Pub. Date: Dec. 8, 2022

(54) METHOD FOR DETECTION OF NUCLEIC ACID SEQUENCES FROM BIOLOGICAL SAMPLES OF MEDICAL, AGRICULTURAL AND BIOTECHNOLOGICAL INTEREST AND APPARATUS THEREOF

(71) Applicant: HI TECHNOLOGIES LTDA., Curitiba - PR (BR)

(72) Inventors: Marcus Vinicius MAZEGA FIGUEREDO, Curitiba - PR (BR); Sergio Renato ROGAL JÚNIOR, Curitiba - PR (BR); Luís Felipe HARTMANN, Curitiba - PR (BR); Adriano Artur TIMM, Curitiba - PR (BR); João Victor PREDEBON, Curitiba - PR (BR); Bruna MEDEIROS DA SILVA, Curitiba - PR (BR); Erika BERGAMO SANTIAGO, Curitiba - PR (BR); Bruna WINKERT RADDATZ, Curitiba - PR (BR); Rafael BENEDETTI, Curitiba - PR (BR)

(73) Assignee: HI TECHNOLOGIES LTDA., Curitiba - PR (BR)

(21) Appl. No.: 17/835,171

(22) Filed: Jun. 8, 2022

(30) Foreign Application Priority Data
Jun. 8, 2021 (BR) 1020210110678

(51) Int. Cl. C12Q 1/70; B01L 3/00; B01L 7/00

(52) U.S. Cl. CPC C12Q 1/70; B01L 3/502715; B01L 7/52; C12Q 1/6851

(57) ABSTRACT

The invention refers to a method of detecting nucleic acid sequences in biological samples from medical, agricultural and biotechnological sources, and a corresponding device, that can be used in health care, in particular in laboratorial diagnosis, to detect genetic sequences, with the objective of identifying viruses and diseases arising from genetic malformations, bringing novelties of using saliva samples, making extraction of RNA from the genetic material from the sample, through a small device with low complexity and innovative design, with the advantages of portable, rapid results, with on-line connectivity to a test results center, dispensing frequent visits to the doctor, hastening the start of treatment, allowing access for groups of people and eliminating the need for a highly qualified operator.

### CROSS-REFERENCE TO RELATED APPLICATIONS AND CLAIM TO PRIORITY

This application is related to Patent Application No. 10 2021 011067 8 filed Jun. 8, 2021, in Brazil, the disclosure of which is incorporated herein by reference and to which priority is claimed.

### FIELD OF THE INVENTION

The invention refers to a method for detecting nucleic acid sequences, from biological samples, with medical, agricultural and biotechnological interest, and a corresponding device, with application in the health area, in particular in laboratorial diagnosis, with the purpose of detecting genetic sequences, with the objective of identifying viruses and diseases arising from genetic malformations and with the advantages of being portable, offering fast results, being accessibly connected to a test results center, dispensing frequent visits to the doctor, hastening the start of treatment, allowing access for groups of people and eliminating the need for a highly qualified operator.

### BACKGROUND OF THE INVENTION

As is known in the field of health care, the detection of nucleic acids is an essential step for several biomedical and biotechnological methodologies, including the detection of agricultural pathogens and human infectious agents. Standard methods, such as PCR (Polymerase Chain Reaction), are effective, however, they require instrumentation and structure that is not portable that limits field applications, such as point-of-care (POC) quick tests. POC quick tests are able to provide quality diagnosis in places without laboratory infrastructure, such as mobile testing points and even in homes, increasing the volume of tests, and therefore are of crucial importance in an epidemiological contingency, especially in a pandemic context, where it is necessary to test-trace-isolate patients effectively.

Acute respiratory syndrome caused by Sars-Cov-2, or simply COVID-19, can be diagnosed through RT-qPCR (Reverse Transcriptase Quantitative PCR), but the global shortage caused by the current pandemic makes difficult the access to equipment and reagents, compromising the diagnosis that is an essential part to contain the disease. This makes essential the development of tests like that of this invention. In addition to the urgent need to create new reliable, scalable and applicable POC methodologies to address COVID-19, it is also necessary to increase testing capacity for other diseases that are already endemic, such as HIV, HBV and HCV, or that may have epidemiological peaks, such as dengue or zika.

The demand for ways to detect nucleic acids with minimal laboratory apparatus has led to the development of several forms of isothermal amplification of nucleic acids, which reduces the complexity and cost of POC equipment. Among these techniques, the most studied and the one with the largest number of commercial kits is loop-mediated isothermal amplification, LAMP.

Current methods for the molecular diagnosis of diseases caused by genetic malformation and the presence of viruses are carried out in clinical laboratories, with complex equipment manipulated by specialists in the health area, using two technical procedures mentioned below:

1) Method of reverse transcription followed by polymerase chain reaction (RT-PCR)—in this method, a sample of RNA is converted into complementary DNA (cDNA) by the process of reverse transcription. The cDNA is then subjected to a molecular technique that involves the use of the polymerase enzyme in a chain reaction, within a controlled environment, with the aim of “amplifying” (reproducing) the genetic material of the collected sample. In amplification, the amount of material is multiplied starting from a small amount of RNA sample, making it possible to analyze and detect the presence of specific stretches of a genetic sequence, such as that of a virus, for example. This method has the disadvantage of using different temperatures in different stages of the process, of needing a long time to reach the result, of needing specialized technicians and of being carried out in complex equipment in laboratories; and

2) Method of reverse transcription followed by loop-mediated isothermal amplification (RT-LAMP)—in this method, a sample of RNA is also converted into complementary DNA (cDNA) by the process of reverse transcription, and, after this step, the cDNA goes through the isothermal amplification process, at a constant temperature between 60° C. and 65° C., and with the use of “primers” that perform the multiplication of the desired genetic material. The result can be obtained by two methods:

2.a) By colorimetry, by changing the color of the reaction medium using pH indicators;

2.b) By fluorescence, by emission of fluorescence using probes conjugated to fluorophores that anneal to the target DNA sequence, or double-stranded DNA intercalating fluorophores; and

2.c) By other methods and mechanisms that detect pH variations, such as electrical, electro-chemical and turbidimetry.

RT-LAMP is simpler than RT-PCR, does not require sophisticated equipment, can be done in localized devices, called “point-of-care” (PoC), and is able to provide results in shorter times.

Currently, there are PoC-type devices that use the RT-LAMP molecular method for disease diagnosis and detection of the presence of viruses from genetic material collected from patients.

In the United States, the FDA has authorized the emergency use of Lucira COVID-19 All-in-One Test Kit. In this PoC device, the patient collects the sample using a nasal swab and applies it to the single tube at the top of a test unit. Only Sars-CoV-2 can be detected, with no application to any other pathogen. The test uses the molecular amplification method (RT-LAMP) for qualitative detection of viral particles in individuals suspected of COVID-19 infection. According to the manufacturer, a positive or negative result is indicated by a green light on top of the test unit after 30 minutes. It is smaller than other available devices, but is disposable, does not have any communication system and does not allow a report to be issued.

OmniLamp, developed through a public-private partnership between Fiocruz (RJ, Brazil) and a company for medical technology research and development, Visuri (MG, Brazil) is described as a PoC system for the detection of SARS-CoV-2 using the RT-LAMP method. This device is not capable of providing any type of pre-amplification treatment of the sample and a negative feature is its physical dimensions.

Searching Brazilian and foreign patent banks the following references were found:

U.S. Pat. No. 10,072,309, entitled “Multiplex method for isothermal detection and identification of nucleic acids of bacteria, viruses and protozoa in real time”, discloses a multiple method of diagnosis by reverse transcription and isothermal loop-mediated amplification (RT-LAMP), to detect, identify and quantify nucleic acids from bacteria, viruses, and protozoa in genetic samples.

In the disclosed method, the diagnosis is made by contacting a sample with two or more sets of isothermal amplification primers, specific for pathogens, and new oligofluorophores, specific for the bacterial, viral, and parasitic nucleic acids target of interest, in sufficient conditions to produce detectable amplification signals in real time in about 10 to 40 minutes. Amplification signals are produced by pathogen-specific fluorogenic markers included in one or more of the primers.

This method has the limitation of being done only in the laboratory and does not have a corresponding point-of-care (PoC) device.

U.S. Pat. No. 9,588,109, entitled “Modular point-of-care devices, systems and corresponding uses”, disclosed diagnostic methods in body fluid analytes, supplied by the patient in cartridges with the appropriate reagents for each diagnosis, for use in PoC devices with the local reading of the analyte and with the sending of data from the reading of the analyte to the laboratory and the return of the diagnosis result to the patient through a public communication network.

This method has the limitation of the diagnostic result being sent to the PoC device and not directly to the patient who needs to wait for the result in the same place where the device is located.

The current molecular methods and PoC devices, used to perform molecular tests for the diagnosis of diseases caused by genetic malformation and the presence of viruses, have the following disadvantages, limitations and inconveniences:

3.a) Diagnostic tests made exclusively for the detection of the genetic sequence of the SARS-COV-2 virus, which causes COVID 19;

3.b) Diagnostic result is indicated only by colorimetry, using a specific pH reagent;

3.c) Sample of genetic material is obtained only with the use of nasal and oropharyngeal swab, which causes discomfort to the patient; and

3.d) Sample preparation is not done by the device, necessary for the nucleic acids to be available for the reverse transcription and amplification steps.

### SUMMARY OF THE INVENTION

The invention was developed to overcome the disadvantages, inconveniences, and limitations of current PoC processes and equipment, through innovation, in order to meet the high demand for the diagnosis of COVID-19, and to carry out nucleic acid detection in biological samples of medical, agricultural, and biotechnological interest. The use of the method claimed herein makes it possible to decentralize the conventional diagnostic system in a safe, practical, and reliable way. In addition, it seeks to make the process of collecting biological samples and obtaining results more dynamic and accessible, since the exam can be performed in medical offices, hospitals, health centers, pharmacies, airports, clinical analysis laboratories and even in shopping centers and corporate offices. This will be possible due to the versatility and easy operation of the equipment combined with the versatility of the RT-LAMP method. The detection method is sensitive and highly reliable. The test result can be viewed by the patient or healthcare professional within 1 hour by SMS or email.

Initially, the intention is to reach as many people as possible to meet the demand generated by the COVID-19 pandemic, seeking to avoid underreporting of cases, as well as allowing professionals to take the necessary measures to avoid exposure of non-contaminated individuals to the virus. In the future, the equipment may be used for molecular tests for the diagnosis of other infectious diseases, with the advantages of being portable, offering quick results, being connected online with the test center, dispensing frequent visits to the doctor, speeding up the start of treatment, allowing access for groups of people and eliminating the need for the operator to be highly qualified.

Some of the technical problems that the prior art do not solve and how the invention solved them include:

4.a) Current technologies use genetic sequence detection only by colorimetry. Solved by the device of the invention with the option of being able to be configured to perform both methodologies, colorimetry and fluorescence, bringing the technical improvement of having a sensor element that supports the reading, both of the color of a pH reagent, as well as the length of wave of the emission of a fluorophore, with just one change in the test kit, being totally transparent to the user as to which analytical model is being used;

4.b) Current technologies adopt sample collection for examination only with the use of nasal and oropharyngeal swabs. Solved by the device of the invention due to the additional possibility of collecting a sample by saliva, bringing the technical improvement of allowing the user to choose a more comfortable way of collecting the sample;

4.c) Current technologies do not allow sample preparation and pre-amplification to be done by the same device. This is a necessary step so that the nucleic acids are available for the enzymes that will do the reverse transcription and amplification. Solved by the device of the invention with the use of its own inlet for this type of sample and the use of a heating cycle of the sample to a programmed temperature and time, bringing the technical improvement of providing a complete solution for carrying out the diagnosis without the need for other equipment to perform this step; and

4.d) Current technologies have the need for the patient to be present at the test site to know the test result. Solved by the device of the invention with the online communication of the device via the Internet with its own server, where the operator will register the patient and monitor the processes that the device is executing. At the end of the diagnosis, the analysts will issue and digitally sign a report for the patient, who will receive a link to remote access on a cell phone or other device, with technical improvement in encryption to ensure the security of sensitive data that are transiting via the Internet, as the device can be used for other genetic samples, such as HIV.

It is an object of the invention to provide a method of detecting nucleic acid sequences in biological samples from medical, agricultural, and biotechnological sources comprising the following steps:

a) Positioning a data card in front of a data reader, and reading a sample heating temperature and a waiting and transition time;

b) Automatically instructing a microprocessor to activate a heating element and keeping it activated until a temperature at a temperature sensor of a thermoblock is equal to the sample heating temperature specified on the data card;

c) With the temperature in the thermoblock at the sample heating temperature, the microprocessor activates an operation indication control board, to request insertion of an extraction tube into a sample analyzer;

d) With the extraction tube inserted into the sample analyzer, the microprocessor keeps the temperature in the thermoblock for the time indicated in the reading of the data card with the reader data;

e) The microprocessor turns off the heating element and turns on a fan for cooling the extraction tube until the temperature indicated by the temperature sensor in the thermoblock is equal to the temperature indicated on a consumable cartridge;

f) With the temperature in the thermoblock at the sample heating temperature, the microprocessor activates the operation indication control board, to indicate the removal of the extraction tube from the sample analyzer;

g) With the removal of the extraction tube from the sample analyzer, the microprocessor activates the heating element and keeps it activated until the temperature indicated by the temperature sensor of the thermoblock is equal to the temperature indicated on the consumable cartridge for this stage of the test;

h) With the temperature in the thermoblock at the sample heating temperature, the microprocessor activates the operation indication control board, to indicate the insertion of the consumable cartridge in the sample analyzer;

i) With the consumable cartridge inserted in the sample analyzer, the microprocessor keeps the temperature in the thermoblock for a time as indicated by the consumable cartridge;

j) After the time indicated by the consumable cartridge is reached, the microprocessor turns off the heating element and activates colorimetry and fluorescence sensors of a temperature control board;

k) The microprocessor reads the test with the colorimetry and fluorescence sensors of the temperature control board;

l) The microprocessor encrypts the data obtained in the test and sends them to a results center via a communication interface; and

m) With the temperature in the thermoblock equal to the temperature indicated by the consumable cartridge for this stage of the test, the microprocessor activates the operation indication control board, to warn the end of the test and removal of the consumable cartridge from the sample analyzer.

It is another object of the invention to provide a method of detecting nucleic acid sequences in biological samples from medical, agricultural, and biotechnological sources, comprising the following steps:

A) An operator registers a patient with a consumable cartridge on a data card;

B) The operator collects a sample of the patient's genetic material;

C) The operator transfers the sample of the collected genetic material to an extraction tube;

D) The operator transfers test data by approaching the data card to a data reader of a sample analyzer;

E) The operator waits for an indication from the sample analyzer that the extraction of the sample of the collected genetic material can be done;

F) The operator inserts the extraction tube with the sample of genetic material collected into an upper hull hole of the sample analyzer;

G) The operator waits for an indication from the sample analyzer that the extraction process is complete;

H) The operator removes the extraction tube from the sample analyzer, divides and transfers the contents of the extraction tube to an internal control tube and to a test tube from the consumable cartridge;

I) The operator inserts the consumable cartridge into the sample analyzer for an amplification process;

J) The operator waits for an indication from the sample analyzer that the amplification process is complete; and

K) The operator removes the consumable cartridge from the sample analyzer and disposes of it in an infectious waste collection.

It is yet another object of the invention to provide an apparatus for carrying out the method of the invention, comprising a structure with a consumable cartridge comprising a predominantly cylindrical trunk shape of small thickness and trapezoidal cross section, with four tubes of tapered cylindrical shape fixed transversally to the cross section, including a test tube, a negative control tube, an internal control tube, and a positive control tube, with a data card attached to the center of a lower part of the consumable cartridge, the data card of the RFID tag type, the consumable cartridge fitted to a sample analyzer by its bottom section; and the sample analyzer formed of two parts: an upper hull and a lower hull; the upper hull of the sample analyzer with a predominantly cylindrical trunk shape, with an approximately triangular shaped cross-section with rounded corners, with ventilation holes of a rectangular shape with rounded corners around an upper face, with a fitting edge around the structure to fit in a channel of the lower hull, internally with two cylindrical structures and with a central hole for fixing screws which fix the cylindrical structures to the lower hull, with the upper face with five circular-shaped holes for the insertion of the tubes according to the diagnostic method, with a first hole for the insertion of the extraction tube and four smaller holes for the insertion of the tubes of the consumable cartridge, with a first smaller hole for the insertion of the negative control tube, a second smaller hole for the insertion of the internal control tube, a third smaller hole for insertion of the test tube and a fourth smaller hole for insertion of the positive control tube; and the lower hull of the sample analyzer with a predominantly cylindrical trunk shape, with an approximately triangular shaped section with rounded corners, with a fitting channel around the structure to fit with the fitting edge of the upper hull of the sample analyzer, with ventilation holes on a lower face in a rectangular shape with rounded corners, the rear face with a cavity in a predominantly rectangular prismatic shape with rounded corners, a cavity with three holes, with a first cavity hole in rectangular shape curved in the lower corners for an input and output serial port, a second cavity hole in rectangular shape for a power input port and a third cavity hole in a circular format for the passage of a service button, and with nine cylindrical columns with a central hole for fixing screws, with a first column and a second column for the screws fixing the lower hull to the upper hull, a third column and a fourth column for the screws fixing the lower hull to the main control board and chassis block, with the third column aligned with a first main control board hole and with a first chassis block hole, with the fourth column aligned with a second main control board hole and with a second chassis block hole, with a fifth column for the screws fixing the lower hull to the chassis block, the fifth column aligned with a third chassis block hole, and with a sixth, seventh, eighth and ninth column to fit a power board, the sixth column of the lower hull aligned with a power board hole.

It is yet another object of the invention to provide an apparatus for carrying out the method of the invention comprising internal electrical and mechanical parts of the sample analyzer comprising: a lighting board, with a circular shape of small thickness, with an upper face and a lower face, with six circular through holes, with a first circular through hole located in the center of the lighting board, a second circular through hole radially located around the first circular through hole and aligned with a thermoblock hole for inserting, lighting and heating the extraction cartridge, with a first LED between the first and second circular through holes, and four other circular through holes radially located around the first circular through hole for insertion and illumination of tubes of the consumable cartridge, with a third circular through hole with a second LED for insertion, illumination and heating of a negative control tube, a fourth circular through hole with a third LED for the insertion, lighting and heating of an internal control tube, a fifth circular through hole with a fourth LED for insertion, lighting and heating of a test tube, and a sixth circular through hole with a fifth LED for insertion, lighting and heating of a positive control tube, the LEDs on the lower face of the lighting board connected with the main control board by a first connector, plug-in or similar, from the lower face of the lighting board, and with four through holes for fixing screws; thermoblock in aluminum, cylindrical in shape, with a top face and a bottom face, with side fins, with a first non-through hole centered in the bottom face for inserting the heating element, a second non-through hole centered on the radial axis of the bottom face for inserting a temperature sensor of the thermoblock and five oblong-shaped holes radially distributed throughout the structure for the insertion of the test tubes, with a first oblong-shaped hole for the insertion of the extraction tube, and the other four oblong-shaped holes located off-center on the top of the thermoblock and in the opposite half to the first oblong-shaped hole for the insertion of the consumable cartridge tubes, with a second oblong-shaped hole for insertion of the negative control tube, a third oblong-shaped hole for insertion of the internal control tube, a fourth oblong-shaped hole for insertion of the test tube and a fifth oblong-shaped hole for insertion of the positive control tube, with a circular-shaped recess and a rectangular shaped recess with four non-through holes for fixing screws on the top face and with eight thermoblock non-through holes for fixing screws on the bottom face, with the heating element of the electrical resistance type or similar, connected to a first connection terminal of the main control board and with the thermoblock temperature sensor of the NTC thermistor type or similar connected to a second connection terminal of the main control board; a small-thickness circular sensor plate, with an upper side and a lower side, with a sensor plate circular hole in the center of the small-thickness circular sensor plate, with four sensor plate through holes for fixing the small-thickness circular sensor plate on the bottom side of the thermoblock, the first sensor plate through hole in line with a first thermoblock non-through hole on the bottom side of the thermoblock, the second sensor plate through hole in line with a second thermoblock non-through hole on the bottom side of the thermoblock, the third sensor plate through hole aligned with a third thermoblock non-through hole on the bottom side of the thermoblock and the fourth sensor plate through hole aligned with a fourth thermoblock non-through hole on the bottom side of the thermoblock, with five colorimetry and fluorescence sensors for the consumable cartridge tubes and for the extraction tube, with a first sensor for the negative control tube, a second sensor for the internal control tube, a third sensor for the test tube, a fourth sensor for the positive control tube, and a fifth sensor for the extraction tube, the sensors connected to the main control board by a second connector, snap-in or similar, from the lower face of the small thickness circular sensor plate a chassis block for fixing the electrical and mechanical parts to each other, with a plastic structure with a mostly cylindrical trunk shape and with two prismatic trunk protrusions that form a flat face facing the front of the apparatus, and the internal structure of the chassis block has cutouts and internal protrusions for fitting and fixing components and passing electrical cables, with a first recessed part with a trapezoid shape on the sides and with a second recessed part in the shape of a rectangle on the flat face, with four chassis block through holes for fixing screws with the thermoblock, a first chassis block through hole) aligned with a first thermoblock non-through hole on the bottom face of the thermoblock and a second chassis block through hole aligned with a second thermoblock non-through hole on the bottom face of the thermoblock, with four cylindrical structures with non-through holes for fixing screws of the fan to the chassis block, and with two chassis block non-through holes and a chassis block through hole for fixing screws with holes in a lower hull of the sample analyzer, the chassis block through hole aligned with a first lower hull hole in the lower hull, a first chassis block non-through hole aligned with a second lower hull hole in the lower hull and a second chassis block non-through hole aligned with a third lower hull hole of the lower hull, with two cylindrical structures with through holes and for fixing screws of the main control board, a first cylindrical structure with through hole aligned with a first main control board hole of the main control board and a second cylindrical structure with through hole aligned with a second main control board hole of the main control board, and with two fixing support through holes for a fixing support of the operation indication control board, a first chassis block hole aligned with a first fixing support hole of the fixing support and a second chassis block hole aligned with a second fixing support hole of the fixing support; a separation skirt made of thermoplastic, trapeze-shaped with rounded corners with chamfered portions, with a chamfered portion at the top of the trapeze-shaped area, two other chamfered portions on the sides of the trapeze-shaped area and two beveled portions at the bottom of the trapeze-shaped area, with a first separation skirt circular hole centered at the top for air passage, four additional separation skirt circular holes for fixing the fan, and an off-center rectangular hole for passing a connection cable from the operation indication control board to a terminal of the main control board, the separation skirt fitted internally to the chassis; the fan is a vane-type cooling fan or similar; the main control board is predominantly rectangular in shape, with diagonal cuts at the two upper ends, with two circular areas for the passage of fixing screws and two recessed areas in a half circle form at the two lower ends, the main control board with the microprocessor, contact terminal bars for connection to the input and output power board, with the data reader of the RFID reader type, with the data reader fixed in a perpendicular position on the top of the main control board with a third connector, plug-in or similar, for connection with the first connector from the lighting board and with a fourth connector, plug-in or similar, for connection with the second connector from the sensor board, the first connection terminal on the bottom of the main control board, of plug-in type or similar, for connection to the heating element of the thermoblock, with the second connection terminal, of plug-in type or similar, for connection of the thermoblock temperature sensor, with a fourth connector for cable connection with the operation indication control board, and with a fifth connector, of plug-in type or similar, for connection to the fan, and with four through holes for fixing screws, a first main control board hole aligned with the third lower hull hole of the chassis block, a second main control board hole in line with the second lower hull hole of the chassis block, a third main control board hole in line with the second cylindrical structure with through hole in the lower hull and a fourth main control board hole in line with the first cylindrical structure with through hole in the lower hull; rectangular-shaped input and output power supply board with four circular-shaped protrusions with through holes for the power board fixing screws to the lower hull, an first inlet and outlet power board hole aligned with a sixth cylindrical frame of the lower hull, a second inlet and outlet power board hole of the inlet and output power board aligned with a seventh cylindrical column of the lower hull, a third inlet and outlet power board hole of the inlet and outlet power board lined up with an eight cylindrical column of the lower hull and a fourth inlet and outlet power board hole of the inlet and outlet power board aligned with a ninth cylindrical column of the lower hull, the power board fitted to the main control board through two contact terminal bars, the input and output power board with an universal-type power input) port for connecting an external power supply, of the voltage-converting and voltage-reducing type, with a USB-type input and output serial port for connecting an external equipment for service access and with a service button; the functioning operation indication control board, with an RGB-type LED, with rectangular shape with two rounded sides, with two holes and for fixing screws, with a fixing support, the fixing support with a predominantly rectangular shape, with a fold forming a structure in the shape of an inclined letter “L”, with a centralized rectangular-shaped hollow area in the upper half of the total area, with two holes and for the plate fixing screws and with two holes for fixing the fixing support in the holes of the chassis block, a first fixing support hole of the fixing support aligned with a first chassis block hole of the chassis block and with a second fixing support hole of fixing support aligned with a second chassis block hole of chassis block.

It is yet another object of the invention to provide an apparatus for carrying out the method of the invention comprising the following external and internal components with the following interconnections: a power supply of the voltage converter and reducer type, unidirectionally connected with a universal type power input port; an input and output power board unidirectionally connected to the power input port and bidirectionally connected with a USB-type input and output serial port; the power input port unidirectionally connected to a main control board and to the power supply; the input and output serial port bidirectionally connected to the main control board; the thermoblock with the heating element of the electrical resistance type or similar, and with the temperature sensor of the NTC thermistor type or similar, both unidirectionally connected to the main control board the operation indication control board unidirectionally connected to the main control board; a lighting board unidirectionally connected to the main control board); the data card of the RFID tag type, unidirectionally connected to the data reader of the RFID reader type, by radio frequency; the data reader bidirectionally connected by electronic link to the main control board and by radio frequency to the data card; the main control board containing the microprocessor of the ESP32 series or similar with internal memory for data and for the dedicated logic program for the operation of the sample analyzer, with special instructions received by reading the data card and that controls the communication of the sample analyzer with other external equipment, with the main control board unidirectionally connected to the power input port, the operation indication control board, the lighting board, the heating element, the temperature sensor and the fan and bidirectionally connected to a sensor board and to the input and output serial port, and with the microprocessor bidirectionally connected to a short-range wireless communication interface and to a Wi-Fi type communication interface; wherein the short-range wireless communication interface is bidirectionally connected by radio frequency to a smartphone or similar device, and bidirectionally connected to the microprocessor; and wherein the Wi-Fi communication interface is bidirectionally connected by radio frequency to one or more computers on the internet cloud or not, and bidirectionally connected to the microprocessor.

These and other objects will be described in further detail below and in the appended drawings.

### BRIEF DESCRIPTION OF THE DRAWINGS

The accompanying drawings illustrate various embodiments of systems, methods, and embodiments of various aspects of the disclosure. A person of ordinary skill in the art will appreciate that the illustrated element boundaries (e.g., boxes, groups of boxes, or other shapes) in the figures represent one example of the various boundaries representative of the disclosed invention. It may be that in some examples one element may be designed as multiple elements or that multiple elements may be designed as one element. In other examples, an element shown as an internal component of one element may be implemented as an external component in another, and vice versa. Furthermore, elements may not be drawn to scale. Non-limiting and non-exhaustive descriptions of the disclosure are described with reference to the following drawings. The components in the figures are not necessarily to scale, emphasis instead being placed upon the illustrated principles.

Accordingly, the various embodiments will hereinafter be described in accordance with the appended drawings, which are provided to illustrate but not to limit the scope of the disclosure in any manner, wherein similar designations denote similar elements, and in which:

FIG. 1, which shows the perspective views of the two parts that form the apparatus of the invention, the consumable cartridge (CCV) and the sample analyzer (AA);

FIG. 2, which shows the front perspective view of the consumable cartridge (CCV) for the apparatus of the invention;

FIG. 3, which shows the top view of the consumable cartridge (CCV) for the apparatus of the invention;

FIG. 4, which shows the bottom perspective view of the consumable cartridge (CCV) for the apparatus of the invention;

FIG. 5, which shows the extraction cartridge (TEX) used in the apparatus of the invention;

FIG. 6, which shows the exploded view of the sample analyzer (AA) for the apparatus of the invention, with the component parts thereof;

FIG. 7, which shows the front view of the upper hull (CS) of the sample analyzer (AA) for the apparatus of the invention;

FIG. 8, which shows the rear view of the upper hull (CS) of the sample analyzer (AA) for the apparatus of the invention;

FIG. 9, which shows the side perspective view of the lower hull (CI) of the sample analyzer (AA) for the apparatus of the invention;

FIG. 10, which shows the bottom view of the lower hull (CI) of the sample analyzer (AA) for the apparatus of the invention;

FIG. 11, which shows the perspective view of the upper face of the lighting board (LD1) with the illuminating LEDs of the tubes used by the sample analyzer (AA) for the apparatus of the invention;

FIG. 12, which shows the perspective view of the bottom side of the lighting board (LD1) with the LEDs for lighting the tubes used by the sample analyzer (AA) for the apparatus of the invention;

FIG. 13, which shows the perspective view of the upper face of the thermoblock (TB) of the sample analyzer (AA) for the apparatus of the invention;

FIG. 14, which shows the perspective view of the lower face of the thermoblock (TB) of the sample analyzer (AA) for the apparatus of the invention;

FIG. 15, which shows the perspective view of the upper face of the sensor plate (PLT) of the sample analyzer (AA) of the apparatus of the invention;

FIG. 16, which shows the perspective view of the lower face of the sensor plate (PLT) of the sample analyzer (AA) for the apparatus of the invention;

FIG. 17, which shows the perspective view of the upper face of the chassis (CH) of the sample analyzer (AA) for the apparatus of the invention;

FIG. 18, which shows the front view of the upper face of the chassis (CH) of the sample analyzer (AA) for the apparatus of the invention;

FIG. 19, which shows the front view of the bottom side of the chassis (CH) of the sample analyzer (AA) for the apparatus of the invention;

FIG. 20, which shows the front view of the front face of the chassis (CH) of the sample analyzer (AA) for the apparatus of the invention;

FIG. 21, which shows the perspective view of the upper face of the separation skirt (SAF) of the sample analyzer (AA) for the apparatus of the invention;

FIG. 22, which shows the front view of the upper face of the separation skirt (SAF) of the sample analyzer (AA) for the apparatus of the invention;

FIG. 23, which shows the perspective view of the top face of the main control board (PPCI) with the data reader (LFC) of the sample analyzer (AA) for the apparatus of the invention;

FIG. 24, which shows the side view of the top face of the main control board (PPCI) with the data reader (LFC) of the sample analyzer (AA) for the apparatus of the invention;

FIG. 25, which shows the front view of the lower face of the main control board (PPCI) of the sample analyzer (AA) for the apparatus of the invention;

FIG. 26, which shows the front view of the upper face of the main control board (PPCI) of the sample analyzer (AA) for the apparatus of the invention;

FIG. 27, which shows the front perspective view of the input and output power board (PA) of the sample analyzer (AA) for the apparatus of the invention;

FIG. 28, which shows the rear view of the input and output power board (PA) of the sample analyzer (AA) for the apparatus of the invention;

FIG. 29, which shows the front view of the lower face of the input and output power board (PA) of the sample analyzer (AA) for the apparatus of the invention;

FIG. 30, which shows the side view of the input and output power board (PA) of the sample analyzer (AA) for the apparatus of the invention;

FIG. 31, which shows the perspective view of the operating indication plate (LD2) of the sample analyzer (AA) for the apparatus of the invention;

FIG. 32, which shows the front view of the operating indication plate (LD2) of the sample analyzer (AA) for the apparatus of the invention;

FIG. 33, which shows the front and side views of the fixing support (SFL) of the operating indication plate (LD2) of the sample analyzer (AA) for the apparatus of the invention;

FIG. 34, which shows the rear perspective view of the fixing support (SFL) of the operating plate (LD2) of the sample analyzer (AA) for the apparatus of the invention;

FIG. 35, which shows the block diagram of the internal components of the sample analyzer (AA) for the device of the invention and its interconnections;

FIG. 36, which shows the block diagram of the process executed by the dedicated microprocessor program (MP), along with the instructions contained in the data card (NFC) of the sample analyzer (AA) for the apparatus of the invention; and

FIG. 37, which shows the block diagram of the process of executing the operator of the apparatus of the invention.

### DETAILED DESCRIPTION OF THE PREFERRED EMBODIMENT(S)

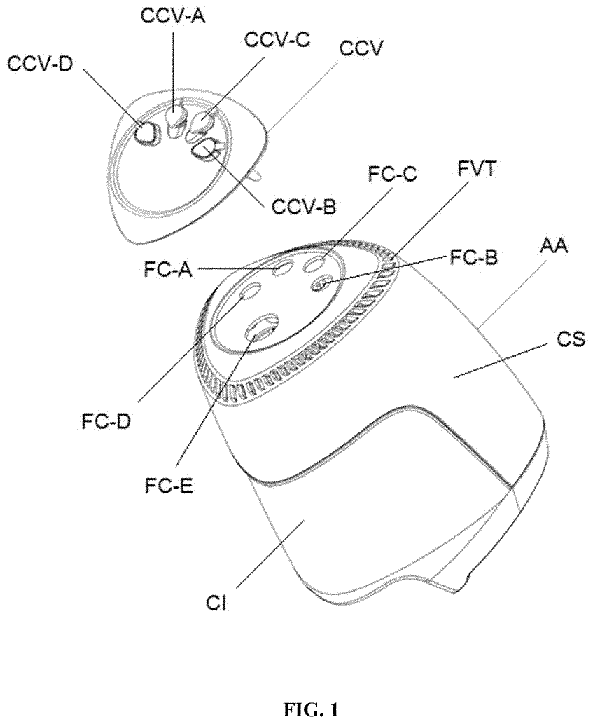

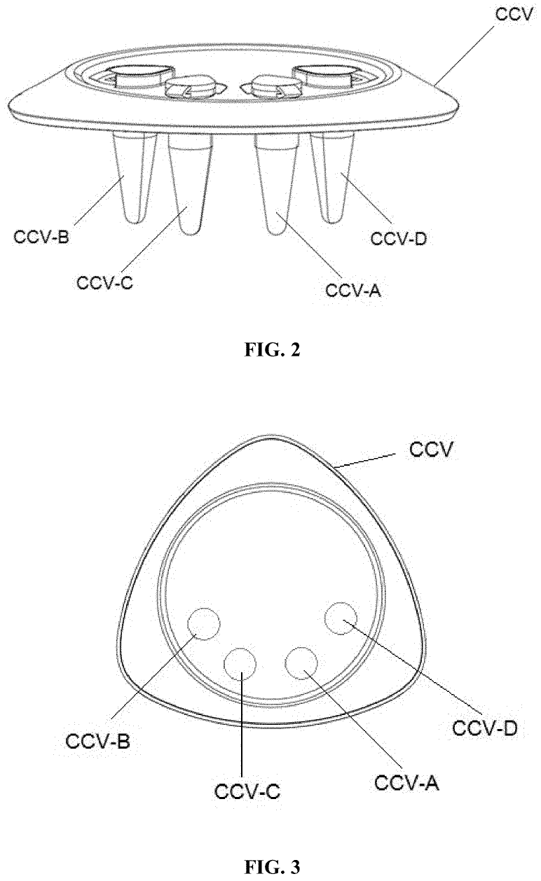

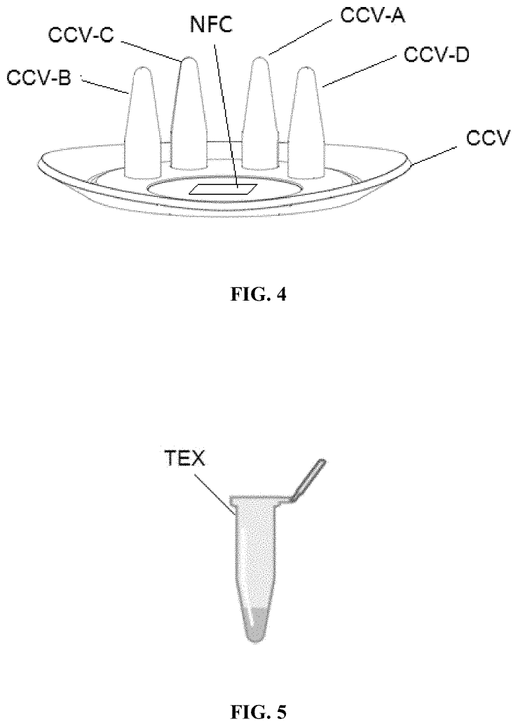

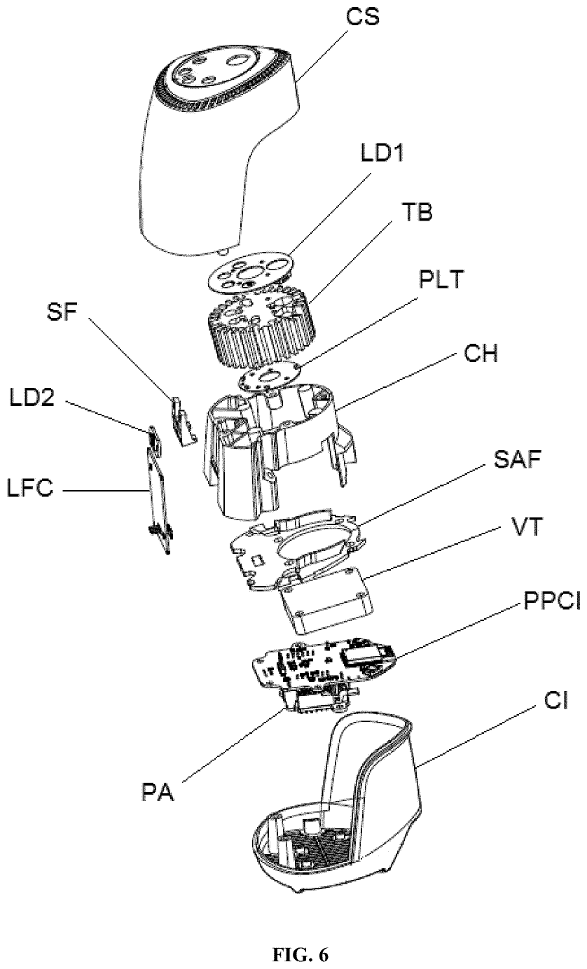

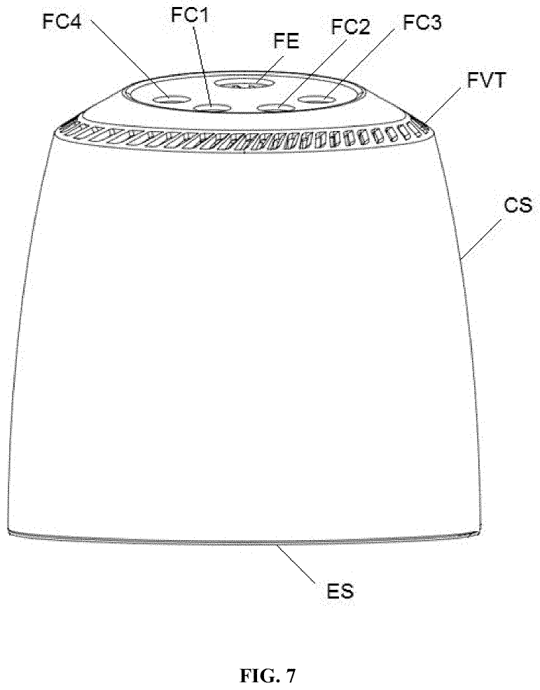

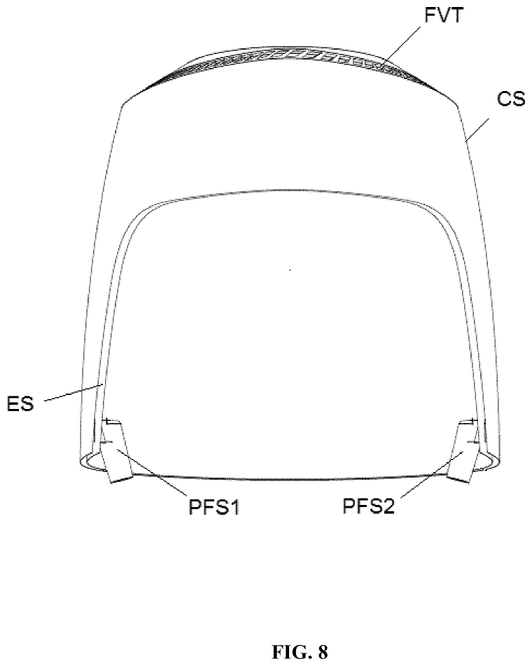

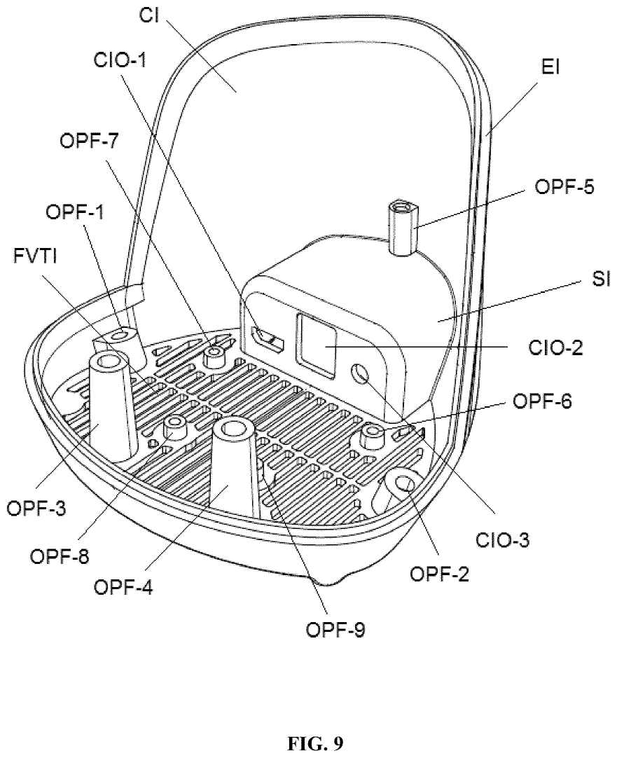

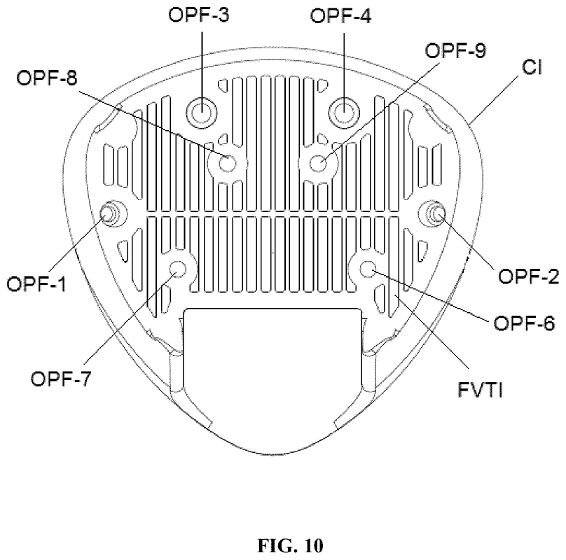

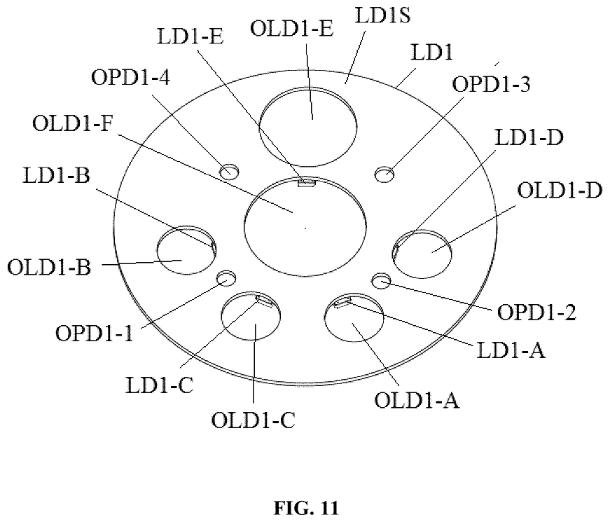

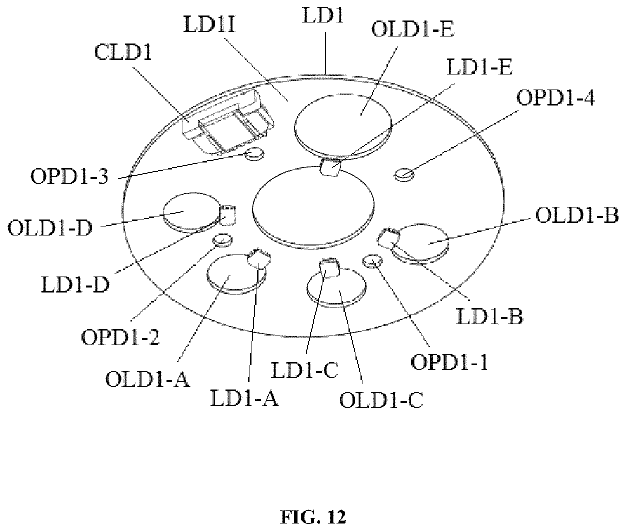

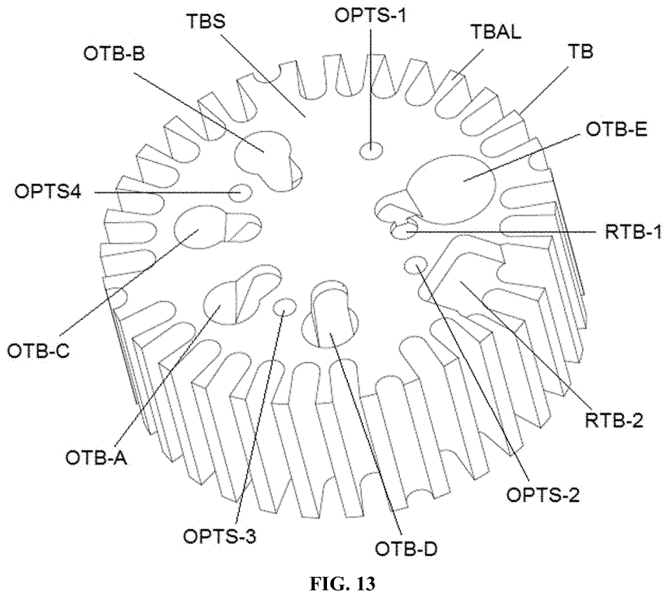

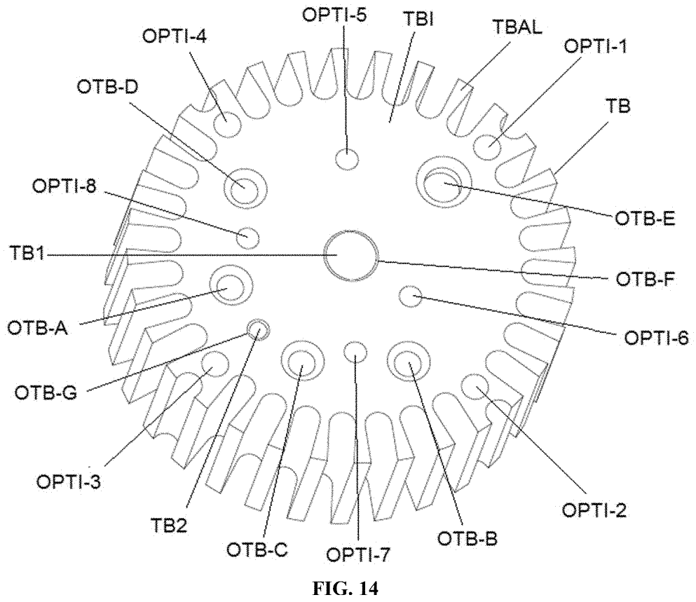

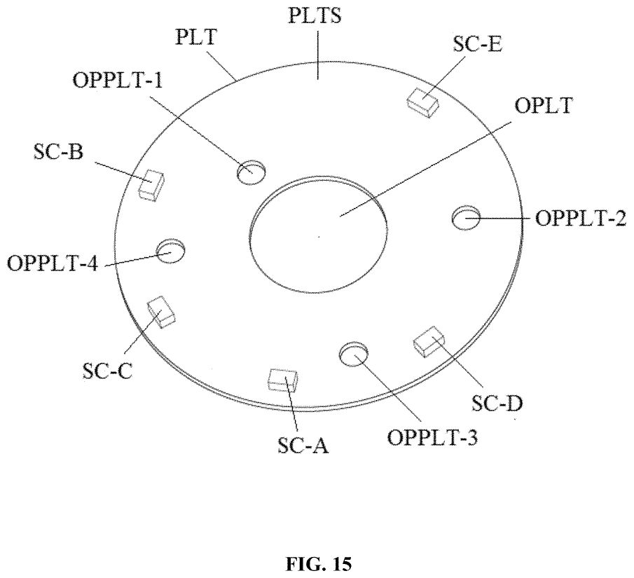

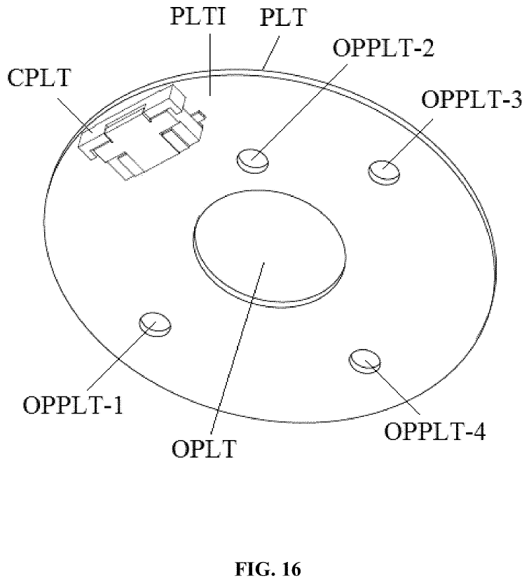

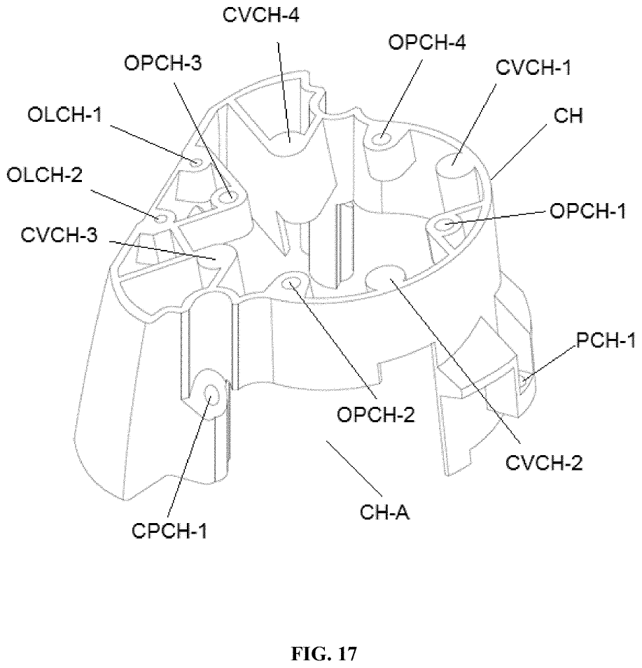

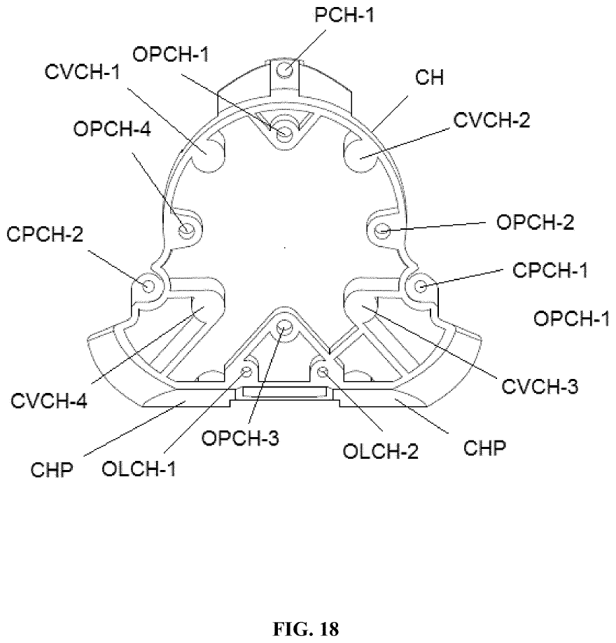

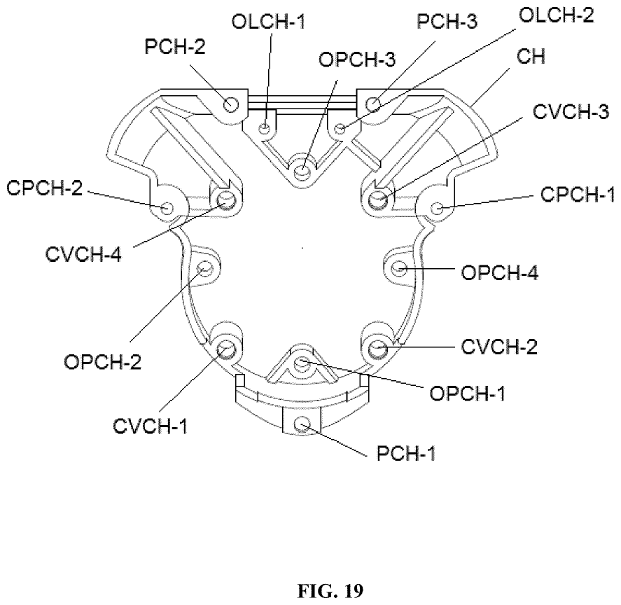

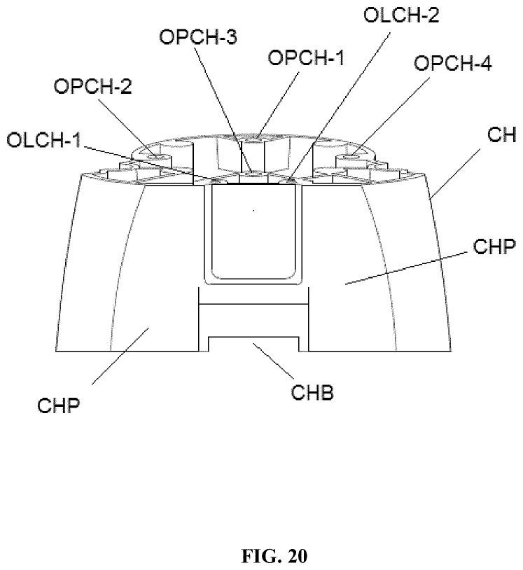

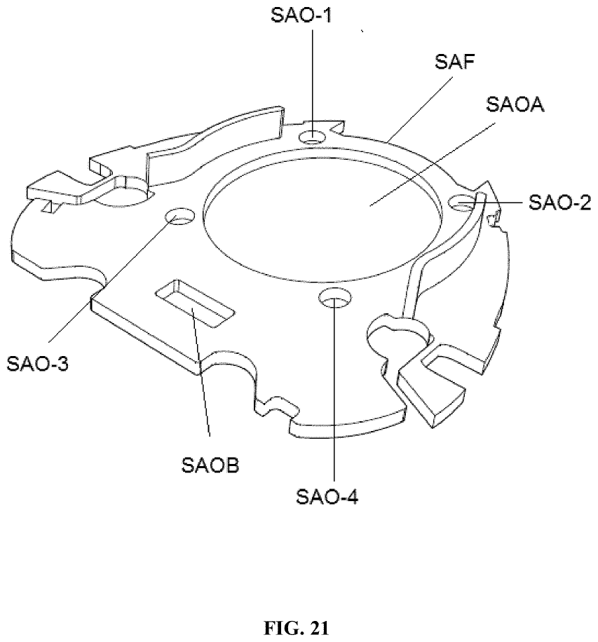

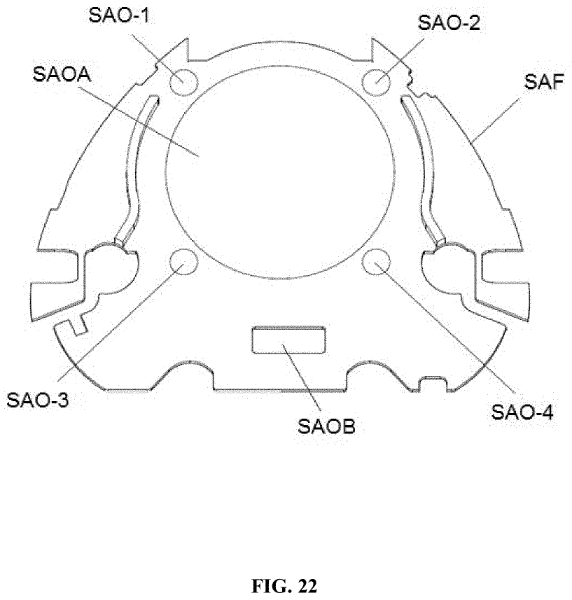

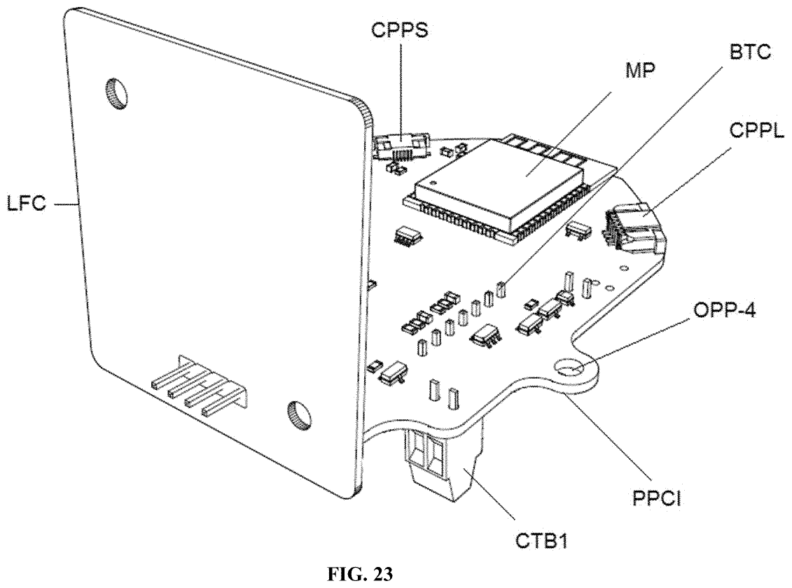

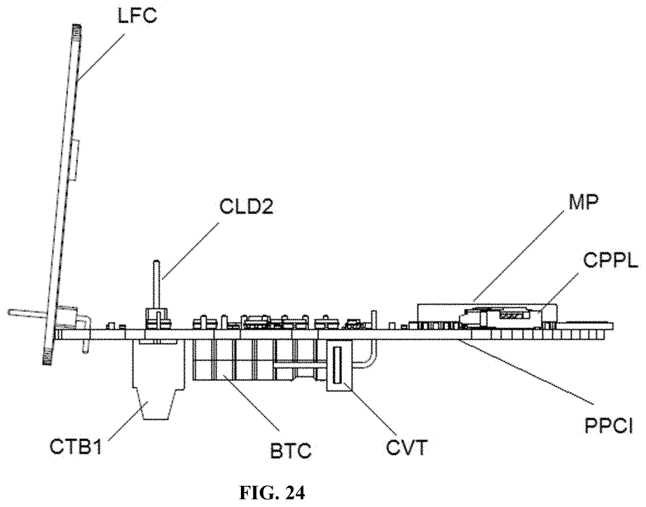

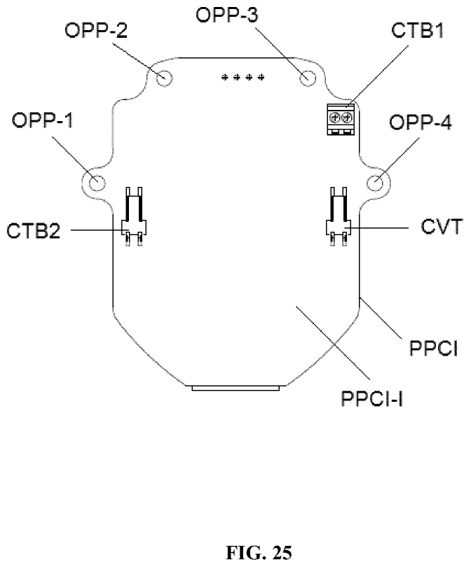

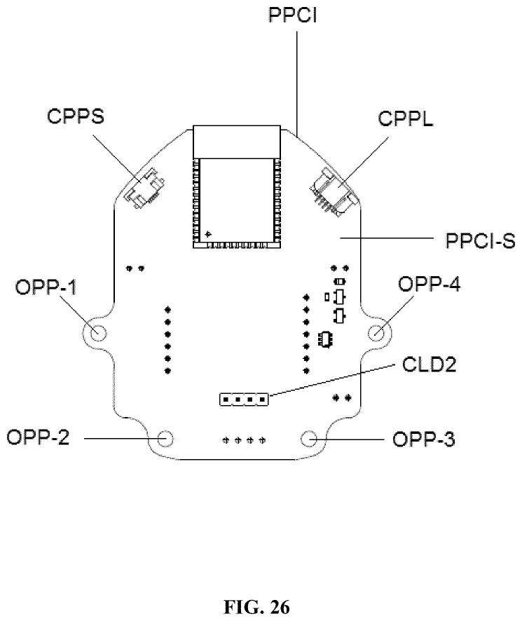

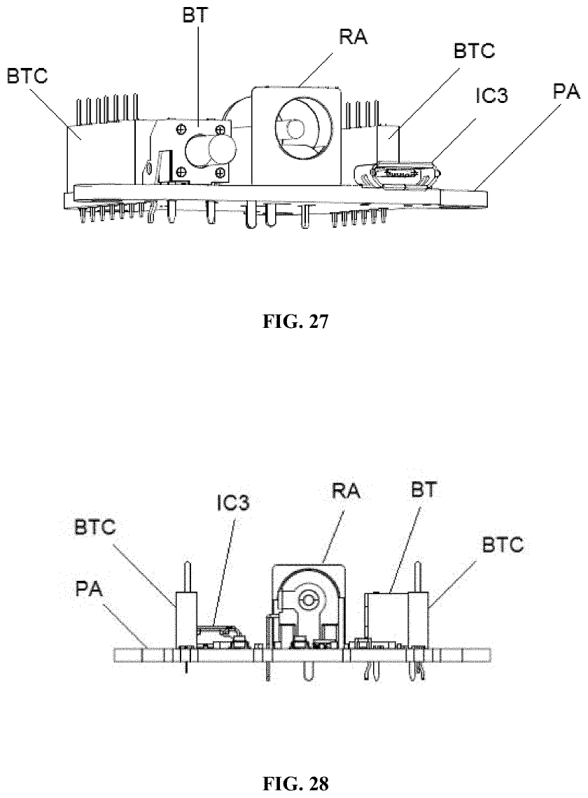

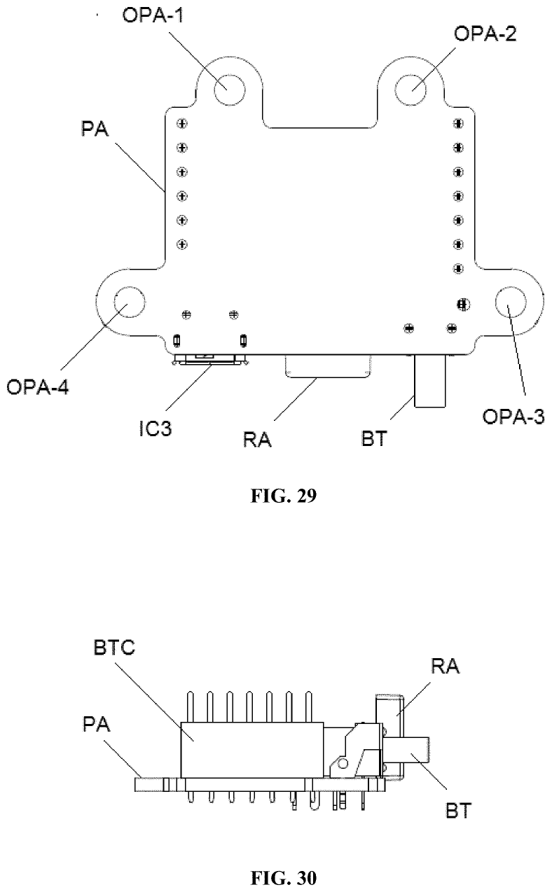

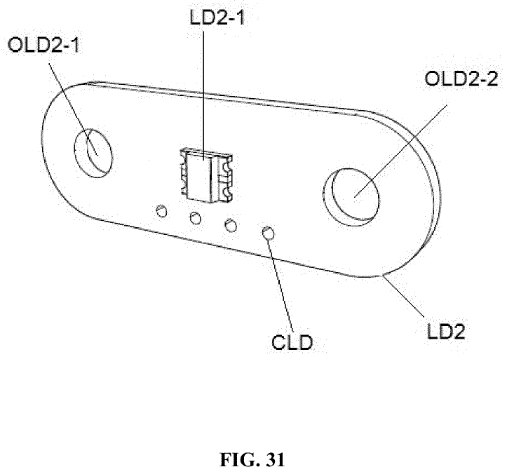

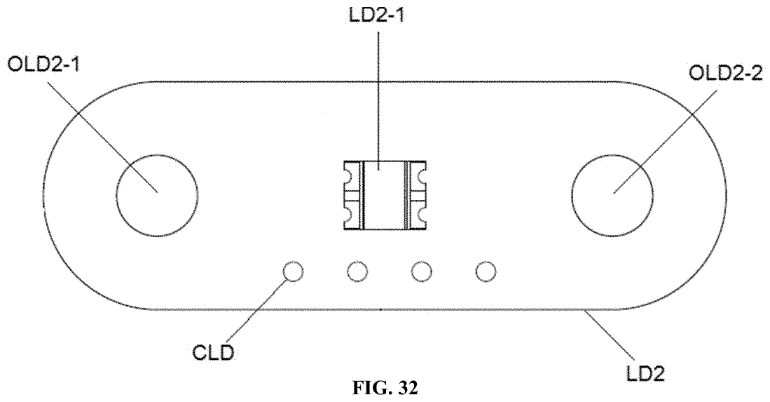

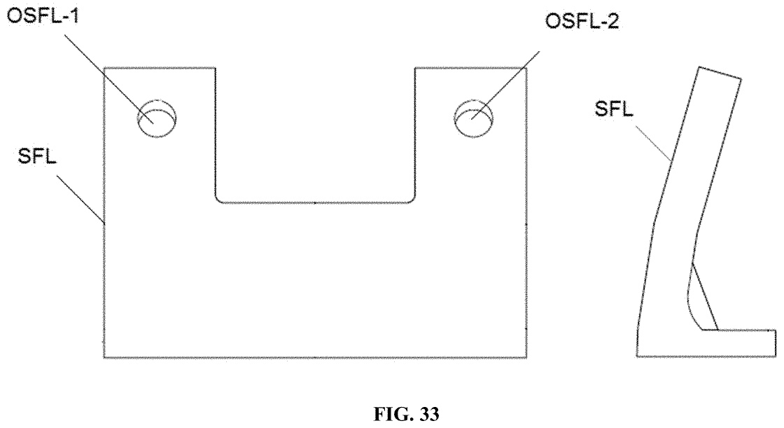

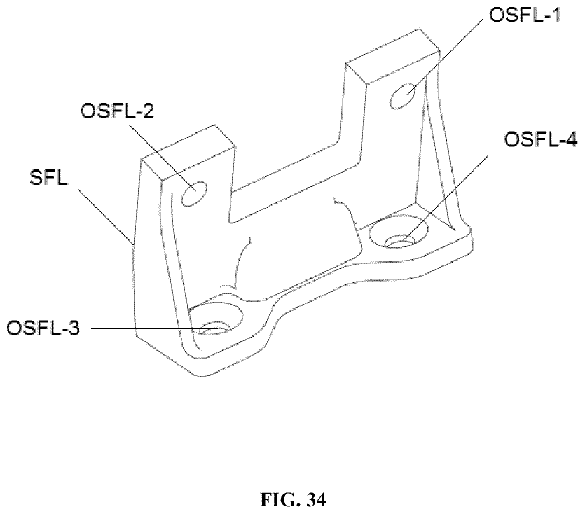

According to FIGS. 1 to 34, the apparatus of the invention is formed of two parts: a consumable cartridge (CCV) and a sample analyzer (AA).

The consumable cartridge (CCV) has a predominantly cylindrical trunk shape of small thickness and trapezoidal cross section, with four tubes of tapered cylindrical shape, fixed transversally to the section, being a test tube (CCV-A), a negative control tube (CCV-B), an internal control tube (CCV-C) and a positive control tube (CCV-D), with the data card (NFC) attached to the center of the lower part, the data card (NFC) of the RFID “tag” type, the consumable cartridge (CCV) being fitted to the sample analyzer (AA) by its bottom section.

The sample analyzer (AA) is formed in its external structure by two parts: the upper hull (CS) and the lower hull (CI); the upper hull (CS) with a predominantly cylindrical trunk shape, with a triangular shape cross-section with rounded corners, with ventilation holes (FVT) of a rectangular shape with rounded corners around the upper face, with a fitting edge (ES) around the structure to fit in the channel (EI) of the lower hull (CI), internally with two cylindrical structures (PFS1) and (PFS2) with a central hole for fixing screws which fix the cylindrical structures to the lower hull (CI), with the upper face with five circular-shaped holes for the insertion of the tubes according to the diagnostic method, being one hole (FC-E) for the insertion of the extraction tube (TEX) and the other four smaller holes for the insertion of the tubes of the consumable cartridge (CCV), with one of the holes (FC-B) for the insertion of the negative control tube (CCV-B), an hole (FC-C) for the insertion of the internal control tube (CCV-C), an hole (FC-A) for insertion of the test tube (CCV-A) and one hole (FC-D) for insertion of the positive control tube (CCV-D).

The sample analyzer (AA) has internal electrical and mechanical parts: a lighting board (LD1), with a circular shape of small thickness, with upper face (LD1S) and lower face (LD1I), with six circular through holes, with a hole (OLD1-F) located in the center of the board, a hole (OLD1-E) radially located around the central hole (OLD1-F) and aligned with the hole (OTB-E) of the thermoblock (TB) for inserting, lighting and heating of the extraction cartridge (TEX), with the LED (LD1-E) between the holes (OLD1-F) and (OLD1-E), and the other four holes radially located around the central hole (OLD1-F), for the insertion and illumination of the tubes of the consumable cartridge (CCV), being a hole (OLD1-B) with the LED (LD1-B), for the insertion, illumination and heating of the tube of negative control (CCV-B), an hole (OLD1-C) with the LED (LD1-C), for the insertion, lighting and heating of the internal control tube (CCV-C), a hole (OLD1-A) with LED (LD1-A), for insertion, lighting and heating of the test tube (CCV-A) and a hole (OLD1-D) with LED (LD1-D), for insertion, lighting and heating of the positive control tube (CCV-D), the LEDs on the bottom side (LD1I) of the lighting board (LD1) connected with the main control board (PPCI) by the connector (CLD1), plug-in or similar, from the bottom side (LD1I) of the lighting board (LD1), and with four through holes (OPD1-1), (OPD1-2), (OPD1-3) and (OPD1-4) for fixing screws.

A thermoblock (TB) in aluminum, cylindrical in shape, with a top face (TBS) and a bottom face (TBI), with side fins (TBAL), with a hole (OTB-F) centered and non-through, in the bottom face (TBI) for inserting the heating element (TB1), a non-through hole (OTB-G) centered on the radial axis of the bottom face, for inserting the temperature sensor (TB2) of the thermoblock (TB) and five holes radially distributed throughout the structure for the insertion of the test tubes, being one hole (OTB-E) for the insertion of the extraction tube (TEX), and the other four holes located off-center on the upper face of the thermoblock (TB) and in the opposite half to the oblong-shaped hole (OTB-E) for the insertion of the consumable cartridge tubes (CCV), being an oblong-shaped hole (OTB-B), and for the insertion of the negative control tube (CCV-B), an oblong-shaped hole (OTB-C), and for the insertion of the internal control tube (CCV-C), an oblong-shaped hole (OTB-A) and for the insertion of the test tube (CCV-A) and an oblong-shaped hole (OTB-D) and for the insertion of the positive control tube (CCV-D), with a recess (RTB-1) with circular shape and one recess (RTB-2) with rectangular shape, with four non-through holes (OPTS-1), (OPTS-2), (OPTS-3) and (OPTS-4) for fixing screws on the top face and with eight non-through holes (OPTI-1), (OPTI-2), (OPTI-3), (OPTI-4), (OPTI-5), (OPTI-6), (OPTI -7) and (OPTI-8), for the fixing screws on the underside, with the heating element (TB1) of the electrical resistance type or similar, connected to the connection terminal (CTB1) of the main control board (PPCI) and with the thermoblock temperature sensor (TB2) of the NTC thermistor type or similar connected to the connection terminal (CTB2) of the main control board (PPCI).

A small-thickness circular sensor plate (PLT), with an upper face (PLTS) and a lower face (PLTI), with a circular hole (OPLT) in the center of the plate, with four through holes (OPPLT-1), (OPPLT-2), (OPPLT-3) and (OPPLT-4) for fixing the plate on the lower face of the thermoblock (TB), the hole (OPPLT-1) in line with the hole (OPTI-5) on the bottom side of the thermoblock (TB), the hole (OPTI-2) in line with the hole (OPTI-6) on the bottom side of the thermoblock (TB), the hole (OPPLT-3) aligned with the hole (OPTI-7) on the bottom side of the thermoblock (TB) and the hole (OPTI-4) aligned with the hole (OPTI-8) on the bottom side of the thermoblock (TB), with five colorimetry and fluorescence sensors for the consumable cartridge tubes (CCV) and for the extraction tube (TEX), being a sensor (SC-B) for the negative control tube (CCV-B), a sensor (SC-C) for the internal control tube (CCV-C), a sensor (SC-A) for the test tube (CCV-A), a sensor (SC-D) for the positive control tube (CCV-D), and a sensor (SC-E) for the extraction tube (TEX), the sensors connected to the main control board (PPCI) by the connector (CPLT), snap-in or similar, from the lower face (PLTI) of the sensor plate (PLT).

A chassis block (CH) for fixing the electrical and mechanical parts to each other, with a plastic structure with a mostly cylindrical trunk shape and with two prismatic trunk protrusions that form a flat face (CHP) facing the front of the device of the invention, and the internal structure of the chassis (CH) has cutouts and internal protrusions for fitting and fixing the components and passing electrical cables, with a recessed part (CH-A) with a trapezoid shape on the sides and with a recessed part (CH-B) in the shape of a rectangle on the flat face (CHP), with four through holes (OPCH-1), (OPCH-2), (OPCH-3) and (OPCH-4) for the fixing screws with the thermoblock (TB), the hole (OPCH-1) aligned with the hole (OTPI-1) of the bottom face (TBI) of the thermoblock (TB) and the hole (OPCH-3) aligned with the hole (OTPI-3) of the bottom face (TBI) of the thermoblock (TB), with four cylindrical structures (CVCH-1), (CVCH-2), (CVCH-3) and (CVCH-4) with non-through holes for the fixing screws of the fan (VT) to the chassis block (CH), and with two non-through holes (PCH-2), (PCH-3) and a through hole (PCH-1) for the fixing screws with the holes in the lower hull (CI), hole (PCH-1) aligned with the hole (OPF-5) in the lower hull (CI), the hole (PCH-2) with the hole (OPF-4) in the lower hull (CI) and the hole (PCH-3) aligned with the hole (OPF-3) of the lower hull (CI), with two cylindrical structures with through holes (CPCH-1) and (CPCH-2) for the fixing screws of the main control board (PPCI), the hole (CPCH-1) aligned with the hole (OPPCI-1) of the main control board (PPCI) and the hole (CPCH-2) aligned with the hole (OPPCI-2) of the main control board (PPCI), and with two through holes for the fixing support (SFL) of the operating indication LED control board (LD2), the hole (OLCH-1) being aligned with the hole (OSFL-4) of the fixing support (SFL) and the hole (OLCH-2) aligned with the hole (OSFL-3) of the fixing support (SFL).

A separation skirt (SAF), made of thermoplastic, trapeze-shaped with rounded corners with chamfered portions, with a chamfered portion at the top of the trapeze area, two other chamfered portions on the sides of the trapeze area and two beveled portions at the bottom of the trapeze area, with a circular hole (SAOA) centered at the top for air passage and four holes (SAO-1), (SAO-2), (SAO-3) and (SAO-4) circular for fixing the fan (VT) and off-center rectangular hole (SAOB) for passing the connection cable from the operating indication LED control board (LD2) to the terminal (CLD2) of the main control board (PPCI), the skirt (SAF) being fitted internally to the chassis (CH).

A vane-type cooling fan (VT) or similar.

A main control board (PPCI), predominantly rectangular in shape, with diagonal cuts at the two upper ends, with two circular areas for the passage of the fixing screws and two recessed areas in a half circle form at the two lower ends, the main control board (PPCI) with the microprocessor (MP), the contact terminal bars (BTC) for connection to the input and output power board (PA), with the RFID-type data reader (LFC), being the data reader (LFC) fixed in a perpendicular position to the main control board (PPCI), the top face (PPCI-S) with a connector (CPPL), plug-in or similar, for connection with the connector (CLD1) from the board (LD1) and with a connector (CPPS), plug-in or similar, for connection with the connector (CPLT) from the sensor board (PLT), the lower face (PPCI-I) with a connector (CTB1), of plug-in type or similar, for connection to the heating element (TB1) of the thermoblock (TB), with a connector (CTB2), of plug-in type or similar, for connection of the thermoblock temperature sensor (TB2), with a connector (CDL2) for cable connection with the operating indication LED control board (LD2), and with a connector (CVT), of plug-in type or similar, for connection to the fan (VT), and with four through holes for fixing screws, the hole (OPP-2) being aligned with the hole (OPF-3) of the chassis block (CH), the hole (OPP-3) in line with the hole (OPF-4) of the chassis block (CH), the hole (OPP-1) in line with the hole (CPCH-2) in the lower hull and the hole (OPP-4) in line with the hole (CPCH-1) in the lower hull.

A rectangular-shaped input and output power supply (PA) board with four circular-shaped protrusions with through holes (OPA-1), (OPA-2), (OPA-3) and (OPA-4) for the power board (PA) fixing screws to the lower hull (CI), the inlet and outlet power board (PA) hole (OPA-3) aligned with the cylindrical frame (OPF-6) of the lower hull (CI), the hole (OPA-4) of the inlet and output power board (PA) aligned with the cylindrical frame (OPF-7) of the lower hull (CI), the hole (OPA-1) of the inlet and outlet power board (PA) lined up with the cylindrical frame (OPF-8) of the lower hull (CI) and the hole (OPA-2) of the inlet and outlet power board (PA) aligned with the cylindrical frame (OPF-9) of the lower hull, the power board (PA) fitted to the main control board (PPCI) through two contact terminal bars (BTC), the input and output power board (PA) with an universal-type power input (RA) port for connecting an external power supply (FA), of the voltage-converting and voltage-reducing type, with a USB-type input and output serial port (IC3) for connecting an external equipment for service access and with a service button (BT).

A functioning indication board (LD2), with the RGB-type LED (LD2-1), with rectangular shape with two rounded sides, with two holes (OLD2-1) and (OLD2-2) for fixing screws, with a fixing support (SFL), the fixing support with a predominantly rectangular shape, with a fold forming a structure in the shape of an inclined letter “L”, with a centralized rectangular-shaped hollow area in the upper half of the total area, with two holes (OSFL-1) and (OSFL-2) for the plate fixing screws (LD2) and with two holes (OSFL-3) and (OSFL-4) for fixing the fixing support (SFL) in the holes (OLCH-1) and (OLCH-2) of the chassis block (CH), the hole (OSFL-4) of the fixing support (SFL) aligned with the hole (OLCH-1) of the chassis block (CH) and with the hole (OSFL-3) of fixing support (SFL) aligned with hole (OLCH-2) of chassis block (CH).

The lower hull (CI) of the sample analyzer (AA) with a predominantly cylindrical trunk shape with an approximately triangular section with rounded corners, with a fitting channel (EI) around the structure to fit with the fitting edge (ES) of the upper hull (CS) of the sample analyzer (AA), with ventilation holes (FVTI) on the lower face in a rectangular shape with rounded corners, the rear face with a cavity (SI) in a predominantly rectangular prismatic shape with rounded corners, the cavity (SI) with three holes, being one hole (CIO-1) in rectangular shape curved in the lower corners for the input and output serial port (IC3), one hole (CIO-2) in rectangular shape for the power input port (RA) and a hole (CIO-3) in a circular format for the passage of the service button (BT), and with nine cylindrical frames with a central hole for the fixing screws, being the frame (OPF-1) and the frame (OPF-2) for the screws fixing the lower hull (CI) to the upper hull (CS), the frame (OPF-3) and the frame (OPF-4) for the screws fixing the lower hull (CI) to the control board (PPCI) and chassis block (CH), being the frame (OPF-3) aligned with the hole (OPP-2) of the main control board (PPCI) and with the hole (PCH-2) of the chassis block, with the frame (OPF-4) aligned with the hole (OPP-3) of the main control board (PPCI) and with the hole (PCH-3) of the chassis block, with the frame (OPF-5) for the screw fixing the lower hull (CI) to the chassis block (CH), the frame (OPF-5) aligned with the hole (PCH-1) of the chassis block (CH), and with four frames (OPF6), (OPF-7), (OPF-8) and (OPF-9) to fit the power board (PA), the frame (OPF-6) of the lower hull aligned with the hole (OPA-3) of the power board (PA).

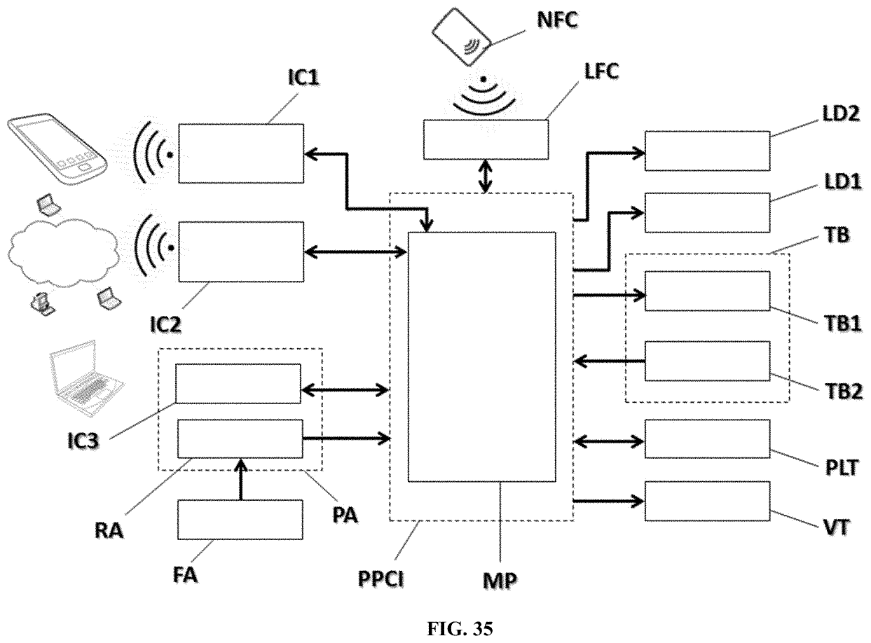

The device of the invention, according to FIG. 35, has the following external and internal components with the following interconnections: power supply (FA), of the voltage converter and reducer type, unidirectionally connected with the universal type power input port (RA); the input and output power board (PA) unidirectionally connected to the power input (RA) port and bidirectionally connected with the USB-type input and output serial port (IC3); power input port (RA) unidirectionally connected to the main control board (PPCI) and to the power supply (FA); the serial port (IC3) bidirectionally connected to the main control board (PPCI); the thermoblock (TB) with the heating element (TB1), of the electrical resistance type or similar, and with the temperature sensor (TB2), of the NTC thermistor type or similar, both unidirectionally connected to the main control board (PPCI); the operating indication board (LD2) unidirectionally connected to the main control board (PPCI); the lighting board (LD1) unidirectionally connected to the main control board (PPCI); data card (NFC) of the RFID Tag type, unidirectionally connected to the data reader (LFC), of the RFID reader type, by radio frequency; data reader (LFC) bidirectionally connected by electronic link to the main control board (PPCI) and by radio frequency to the data card (NFC); the main control board (PPCI) containing the microprocessor (MP) of the ESP32 series or similar, with internal memory for data and for the dedicated logic program for the operation of the sample analyzer (AA), with special instructions received by reading the data card (NFC) and that controls the communication of the sample analyzer (AA) with other external equipment, being the board (PPCI) unidirectionally connected to the input port (RA), to the operation indication board (LD2), to the lighting board (LD1), to the heating element (TB1), to the thermoblock temperature sensor (TB2) and to the fan (VT) and bidirectionally connected to the sensor board (PLT) and to the serial port (IC3), and being the microprocessor (MP) bidirectionally connected to the communication interface (IC1), of the Bluetooth® type, and to the communication interface (IC2), of the WIFI type; communication interface (ICI) bidirectionally connected by radio frequency, to a smartphone or similar device, and bidirectionally connected to the microprocessor (MP); communication interface (IC2) bidirectionally connected by radio frequency to one or more computers, in the Internet cloud or not, and bidirectionally connected to the microprocessor (MP).

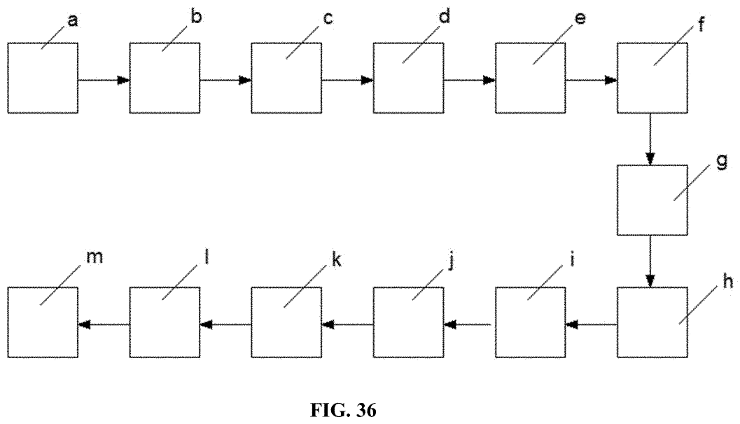

The process performed with the aid of dedicated software stored in the microprocessor memory (MP) of the device of the invention and the special instructions provided on the data card (NFC) takes place in the following sequence, shown in FIG. 36:

a) When positioning the data card (NFC) in front of the data reader (LFC), the sample heating temperature and the waiting and transition times are read;

b) The microprocessor (MP) automatically activates the heating element (TB1) and keeps it activated until the temperature at the temperature sensor (TB2) of the thermoblock (TB) is equal to the temperature specified on the data card (NFC);

c) With the temperature in the thermoblock (TB) at the desired value, the microprocessor (MP) activates the operating indication board (LD2), to request the insertion of the extraction tube (TEX) in the sample analyzer (AA);

d) With the extraction tube (TEX) inserted into the sample analyzer (AA), the microprocessor (MP) keeps the temperature in the thermoblock (TB) for the time indicated in the reading of the data card (NFC) with the reader data (LFC);

e) The microprocessor (MP) turns off the heating element (TB1) and turns on the fan (VT) for cooling the extraction tube (TEX) until the temperature indicated by the temperature sensor (TB2) in the thermoblock (TB) is equal to the temperature indicated on the consumable cartridge (CCV);

f) With the temperature in the thermoblock (TB) at the desired value, the microprocessor (MP) activates the operation indication control board (LD2), to indicate the removal of the extraction tube (TEX) from the sample analyzer (AA);

g) With the removal of the extraction tube (TEX) from the sample analyzer (AA), the microprocessor (MP) activates the heating element (TB1) and keeps it activated until the temperature indicated by the temperature sensor (TB2) of the thermoblock (TB) is equal to the temperature indicated on the consumable cartridge (CCV) for this stage of the test;

h) With the temperature in the thermoblock (TB) at the desired value, the microprocessor (MP) activates the operation indication board (LD2), to indicate the insertion of the consumable cartridge (CCV) in the sample analyzer (AA);

i) With the consumable cartridge (CCV) inserted in the sample analyzer (AA), the microprocessor (MP) keeps the temperature in the thermoblock (TB) by the time as indicated by the consumable cartridge (CCV);

j) After the time indicated by the consumable cartridge (CCV), the microprocessor (MP) turns off the heating element (TB1) and activates the colorimetry and fluorescence sensors of the temperature control board (PLT);

k) The microprocessor (MP) reads the test with the colorimetry and fluorescence sensors of the sensor board (PLT);

l) The microprocessor (MP) encrypts the data obtained in the test and sends them to a results center via the communication interface (IC1), or (IC2) or (IC3); and

m) With the temperature in the thermoblock (TB) equal to the temperature indicated by the consumable cartridge (CCV) for this stage of the test, the microprocessor activates the operating indication board (LD2), to warn the end of the test and removing the consumable cartridge (CCV) from the sample analyzer (AA).

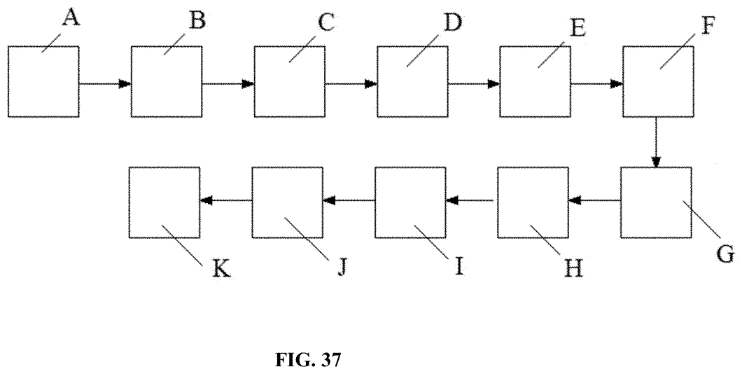

The device operator's execution process takes place in the following sequence, as shown in FIG. 37:

A) The operator registers the patient with the consumable cartridge (CCV) data on the data card (NFC);

B) The operator collects the sample of the patient's genetic material;

C) The operator transfers the sample of the collected genetic material to the extraction tube (TEX);

D) The operator transfers the test data by approaching the data card (NFC) to the data reader (LFC) of the sample analyzer (AA);

E) The operator waits for the indication made by the sample analyzer (AA) that the extraction of the sample of the collected genetic material can be done;

F) The operator inserts the extraction tube (TEX) with the sample of genetic material collected into the hole (FE) of the upper hull (CS) of the sample analyzer (AA);

G) The operator waits for the indication from the sample analyzer (AA) that the extraction process is complete;

H) The operator removes the extraction tube (TEX) from the sample analyzer (AA), divides and transfers the contents of the extraction tube (TEX) to the internal control tube (CCV-C) and to the test tube (CCV-A) from the consumable cartridge (CCV);

I) The operator inserts the consumable cartridge (CCV) into the sample analyzer (AA) for the amplification process;

J) The operator awaits for the indication by the sample analyzer (AA) that the amplification process is complete; and

K) The operator removes the consumable cartridge (CCV) from the sample analyzer (AA) and disposes of it in the infectious waste collection.

While there is shown and described herein certain specific structures illustrating various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

1. A method of detecting nucleic acid sequences in biological samples from medical, agricultural, and biotechnological sources comprising the following steps:
a) Positioning a data card in front of a data reader, and reading a sample heating temperature and a waiting and transition time;
b) Automatically instructing a microprocessor to activate a heating element and keeping it activated until a temperature at a temperature sensor of a thermoblock is equal to the sample heating temperature specified on the data card;
c) With the temperature in the thermoblock at the sample heating temperature, the microprocessor activates an operation indication control board, to request insertion of an extraction tube into a sample analyzer;
d) With the extraction tube inserted into the sample analyzer, the microprocessor keeps the temperature in the thermoblock for the time indicated in the reading of the data card with the reader data;
e) The microprocessor turns off the heating element and turns on a fan for cooling the extraction tube until the temperature indicated by the temperature sensor in the thermoblock is equal to the temperature indicated on a consumable cartridge;
f) With the temperature in the thermoblock at the sample heating temperature, the microprocessor activates the operation indication control board, to indicate the removal of the extraction tube from the sample analyzer;
g) With the removal of the extraction tube from the sample analyzer, the microprocessor activates the heating element and keeps it activated until the temperature indicated by the temperature sensor of the thermoblock is equal to the temperature indicated on the consumable cartridge for this stage of the test;
h) With the temperature in the thermoblock at the sample heating temperature, the microprocessor activates the operation indication control board, to indicate the insertion of the consumable cartridge in the sample analyzer;
i) With the consumable cartridge inserted in the sample analyzer, the microprocessor keeps the temperature in the thermoblock for a time as indicated by the consumable cartridge;
j) After the time indicated by the consumable cartridge is reached, the microprocessor turns off the heating element and activates colorimetry and fluorescence sensors of a temperature control board;
k) The microprocessor reads the test with the colorimetry and fluorescence sensors of the temperature control board;
l) The microprocessor encrypts the data obtained in the test and sends them to a results center via a communication interface; and
m) With the temperature in the thermoblock equal to the temperature indicated by the consumable cartridge for this stage of the test, the microprocessor activates the operation indication control board, to warn the end of the test and removal of the consumable cartridge from the sample analyzer.

2. A method of detecting nucleic acid sequences in biological samples from medical, agricultural, and biotechnological sources, comprising the following steps:
A) An operator registers a patient with a consumable cartridge on a data card;
B) The operator collects a sample of the patient's genetic material;
C) The operator transfers the sample of the collected genetic material to an extraction tube;
D) The operator transfers test data by approaching the data card to a data reader of a sample analyzer;
E) The operator waits for an indication from the sample analyzer that the extraction of the sample of the collected genetic material can be done;
F) The operator inserts the extraction tube with the sample of genetic material collected into an upper hull hole of the sample analyzer;
G) The operator waits for an indication from the sample analyzer that the extraction process is complete;
H) The operator removes the extraction tube from the sample analyzer, divides and transfers the contents of the extraction tube to an internal control tube and to a test tube from the consumable cartridge;
I) The operator inserts the consumable cartridge into the sample analyzer for an amplification process;
J) The operator waits for an indication from the sample analyzer that the amplification process is complete; and
K) The operator removes the consumable cartridge from the sample analyzer and disposes of it in an infectious waste collection.

3. An apparatus for carrying out the method of claim 1, comprising a structure with a consumable cartridge comprising a predominantly cylindrical trunk shape of small thickness and trapezoidal cross section, with four tubes of tapered cylindrical shape fixed transversally to the cross section, including a test tube, a negative control tube, an internal control tube, and a positive control tube, with a data card attached to the center of a lower part of the consumable cartridge, the data card of the RFID tag type, the consumable cartridge fitted to a sample analyzer by its bottom section; and the sample analyzer formed of two parts: an upper hull and a lower hull; the upper hull of the sample analyzer with a predominantly cylindrical trunk shape, with an approximately triangular shaped cross-section with rounded corners, with ventilation holes of a rectangular shape with rounded corners around an upper face, with a fitting edge around the structure to fit in a channel of the lower hull, internally with two cylindrical structures and with a central hole for fixing screws which fix the cylindrical structures to the lower hull, with the upper face with five circular-shaped holes for the insertion of the tubes according to the diagnostic method, with a first hole for the insertion of the extraction tube and four smaller holes for the insertion of the tubes of the consumable cartridge, with a first smaller hole for the insertion of the negative control tube, a second smaller hole for the insertion of the internal control tube, a third smaller hole for insertion of the test tube and a fourth smaller hole for insertion of the positive control tube; and the lower hull of the sample analyzer with a predominantly cylindrical trunk shape, with an approximately triangular shaped section with rounded corners, with a fitting channel around the structure to fit with the fitting edge of the upper hull of the sample analyzer, with ventilation holes on a lower face in a rectangular shape with rounded corners, the rear face with a cavity in a predominantly rectangular prismatic shape with rounded corners, a cavity with three holes, with a first cavity hole in rectangular shape curved in the lower corners for an input and output serial port, a second cavity hole in rectangular shape for a power input port and a third cavity hole in a circular format for the passage of a service button, and with nine cylindrical columns with a central hole for fixing screws, with a first column and a second column for the screws fixing the lower hull to the upper hull, a third column and a fourth column for the screws fixing the lower hull to the main control board and chassis block, with the third column aligned with a first main control board hole and with a first chassis block hole, with the fourth column aligned with a second main control board hole and with a second chassis block hole, with a fifth column for the screws fixing the lower hull to the chassis block, the fifth column aligned with a third chassis block hole, and with a sixth, seventh, eighth and ninth column to fit a power board, the sixth column of the lower hull aligned with a power board hole.

4. An apparatus for carrying out the method of claim 1 comprising internal electrical and mechanical parts of the sample analyzer comprising: a lighting board, with a circular shape of small thickness, with an upper face and a lower face, with six circular through holes, with a first circular through hole located in the center of the lighting board, a second circular through hole radially located around the first circular through hole and aligned with a thermoblock hole for inserting, lighting and heating the extraction cartridge, with a first LED between the first and second circular through holes, and four other circular through holes radially located around the first circular through hole for insertion and illumination of tubes of the consumable cartridge, with a third circular through hole with a second LED for insertion, illumination and heating of a negative control tube, a fourth circular through hole with a third LED for the insertion, lighting and heating of an internal control tube, a fifth circular through hole with a fourth LED for insertion, lighting and heating of a test tube, and a sixth circular through hole with a fifth LED for insertion, lighting and heating of a positive control tube, the LEDs on the lower face of the lighting board connected with the main control board by a first connector, plug-in or similar, from the lower face of the lighting board, and with four through holes for fixing screws; thermoblock in aluminum, cylindrical in shape, with a top face and a bottom face, with side fins, with a first non-through hole centered in the bottom face for inserting the heating element, a second non-through hole centered on the radial axis of the bottom face for inserting a temperature sensor of the thermoblock and five oblong-shaped holes radially distributed throughout the structure for the insertion of the test tubes, with a first oblong-shaped hole for the insertion of the extraction tube, and the other four oblong-shaped holes located off-center on the top of the thermoblock and in the opposite half to the first oblong-shaped hole for the insertion of the consumable cartridge tubes, with a second oblong-shaped hole for insertion of the negative control tube, a third oblong-shaped hole for insertion of the internal control tube, a fourth oblong-shaped hole for insertion of the test tube and a fifth oblong-shaped hole for insertion of the positive control tube, with a circular-shaped recess and a rectangular shaped recess with four non-through holes for fixing screws on the top face and with eight thermoblock non-through holes for fixing screws on the bottom face, with the heating element of the electrical resistance type or similar, connected to a first connection terminal of the main control board and with the thermoblock temperature sensor of the NTC thermistor type or similar connected to a second connection terminal of the main control board; a small-thickness circular sensor plate, with an upper side and a lower side, with a sensor plate circular hole in the center of the small-thickness circular sensor plate, with four sensor plate through holes for fixing the small-thickness circular sensor plate on the bottom side of the thermoblock, the first sensor plate through hole in line with a first thermoblock non-through hole on the bottom side of the thermoblock, the second sensor plate through hole in line with a second thermoblock non-through hole on the bottom side of the thermoblock, the third sensor plate through hole aligned with a third thermoblock non-through hole on the bottom side of the thermoblock and the fourth sensor plate through hole aligned with a fourth thermoblock non-through hole on the bottom side of the thermoblock, with five colorimetry and fluorescence sensors for the consumable cartridge tubes and for the extraction tube, with a first sensor for the negative control tube, a second sensor for the internal control tube, a third sensor for the test tube, a fourth sensor for the positive control tube, and a fifth sensor for the extraction tube, the sensors connected to the main control board by a second connector, snap-in or similar, from the lower face of the small thickness circular sensor plate a chassis block for fixing the electrical and mechanical parts to each other, with a plastic structure with a mostly cylindrical trunk shape and with two prismatic trunk protrusions that form a flat face facing the front of the apparatus, and the internal structure of the chassis block has cutouts and internal protrusions for fitting and fixing components and passing electrical cables, with a first recessed part with a trapezoid shape on the sides and with a second recessed part in the shape of a rectangle on the flat face, with four chassis block through holes for fixing screws with the thermoblock, a first chassis block through hole) aligned with a first thermoblock non-through hole on the bottom face of the thermoblock and a second chassis block through hole aligned with a second thermoblock non-through hole on the bottom faceof the thermoblock, with four cylindrical structures with non-through holes for fixing screws of the fan to the chassis block, and with two chassis block non-through holes and a chassis block through hole for fixing screws with holes in a lower hull of the sample analyzer, the chassis block through hole aligned with a first lower hull hole in the lower hull, a first chassis block non-through hole aligned with a second lower hull hole in the lower hull and a second chassis block non-through hole aligned with a third lower hull hole of the lower hull, with two cylindrical structures with through holes and for fixing screws of the main control board, a first cylindrical structure with through hole aligned with a first main control board hole of the main control board and a second cylindrical structure with through hole aligned with a second main control board hole of the main control board, and with two fixing support through holes for a fixing support of the operation indication control board, a first chassis block hole aligned with a first fixing support hole of the fixing support and a second chassis block hole aligned with a second fixing support hole of the fixing support; a separation skirt made of thermoplastic, trapeze-shaped with rounded corners with chamfered portions, with a chamfered portion at the top of the trapeze-shaped area, two other chamfered portions on the sides of the trapeze-shaped area and two beveled portions at the bottom of the trapeze-shaped area, with a first separation skirt circular hole centered at the top for air passage, four additional separation skirt circular holes for fixing the fan, and an off-center rectangular hole for passing a connection cable from the operation indication control board to a terminal of the main control board, the separation skirt fitted internally to the chassis; the fan is a vane-type cooling fan or similar; the main control board is predominantly rectangular in shape, with diagonal cuts at the two upper ends, with two circular areas for the passage of fixing screws and two recessed areas in a half circle form at the two lower ends, the main control board with the microprocessor, contact terminal bars for connection to the input and output power board, with the data reader of the RFID reader type, with the data reader fixed in a perpendicular position on the top of the main control board with a third connector, plug-in or similar, for connection with the first connector from the lighting board and with a fourth connector, plug-in or similar, for connection with the second connectorfrom the sensor board, the first connection terminal on the bottom of the main control board, of plug-in type or similar, for connection to the heating element of the thermoblock, with the second connection terminal, of plug-in type or similar, for connection of the thermoblock temperature sensor, with a fourth connector for cable connection with the operation indication control board, and with a fifth connector, of plug-in type or similar, for connection to the fan, and with four through holes for fixing screws, a first main control board hole aligned with the third lower hull hole of the chassis block, a second main control board hole in line with the second lower hull hole of the chassis block, a third main control board hole in line with the second cylindrical structure with through hole in the lower hull and a fourth main control board hole in line with the first cylindrical structure with through hole in the lower hull; rectangular-shaped input and output power supply board with four circular-shaped protrusions with through holes for the power board fixing screws to the lower hull, an first inlet and outlet power board hole aligned with a sixth cylindrical frame of the lower hull, a second inlet and outlet power board hole of the inlet and output power board aligned with a seventh cylindrical column of the lower hull, a third inlet and outlet power board hole of the inlet and outlet power board lined up with an eight cylindrical column of the lower hull and a fourth inlet and outlet power board hole of the inlet and outlet power board aligned with a ninth cylindrical column of the lower hull, the power board fitted to the main control board through two contact terminal bars, the input and output power board with an universal-type power input) port for connecting an external power supply, of the voltage-converting and voltage-reducing type, with a USB-type input and output serial port for connecting an external equipment for service access and with a service button; the functioning operation indication control board, with an RGB-type LED, with rectangular shape with two rounded sides, with two holes and for fixing screws, with a fixing support, the fixing support with a predominantly rectangular shape, with a fold forming a structure in the shape of an inclined letter “L”, with a centralized rectangular-shaped hollow area in the upper half of the total area, with two holes and for the plate fixing screws and with two holes for fixing the fixing support in the holes of the chassis block, a first fixing support hole of the fixing support aligned with a first chassis block hole of the chassis block and with a second fixing support hole of fixing support aligned with a second chassis block hole of chassis block.

5. An apparatus for carrying out the method of claim 1 comprising the following external and internal components with the following interconnections: a power supply of the voltage converter and reducer type, unidirectionally connected with a universal type power input port; an input and output power board unidirectionally connected to the power input port and bidirectionally connected with a USB-type input and output serial port; the power input port unidirectionally connected to a main control board and to the power supply; the input and output serial port bidirectionally connected to the main control board; the thermoblock with the heating element of the electrical resistance type or similar, and with the temperature sensor of the NTC thermistor type or similar, both unidirectionally connected to the main control board the operation indication control board unidirectionally connected to the main control board; a lighting board unidirectionally connected to the main control board); the data card of the RFID tag type, unidirectionally connected to the data reader of the RFID reader type, by radio frequency; the data reader bidirectionally connected by electronic link to the main control board and by radio frequency to the data card; the main control board containing the microprocessor of the ESP32 series or similar with internal memory for data and for the dedicated logic program for the operation of the sample analyzer, with special instructions received by reading the data card and that controls the communication of the sample analyzer with other external equipment, with the main control board unidirectionally connected to the power input port, the operation indication control board, the lighting board, the heating element, the temperature sensor and the fan and bidirectionally connected to a sensor board and to the input and output serial port, and with the microprocessor bidirectionally connected to a short-range wireless communication interface and to a Wi-Fi type communication interface; wherein the short-range wireless communication interface is bidirectionally connected by radio frequency to a smartphone or similar device, and bidirectionally connected to the microprocessor; and wherein the Wi-Fi communication interface is bidirectionally connected by radio frequency to one or more computers on the internet cloud or not, and bidirectionally connected to the microprocessor.
